(19) United States
(12) Patent Application Publication
(10) Pub. No.: US 2012/0156139 A1
(43) Pub. Date: Jun. 21, 2012

(54) ISOTOPICALLY LABELED NEUROCHEMICAL AGENTS AND USES THEROF FOR DIAGNOSING CONDITIONS AND DISORDERS

(76) Inventor: Rachel Katz-Brull, Modi'in-Maccabim-Re'ut (IL)

(21) Appl. No.: 13/392,979

(22) PCT Filed: Jan. 14, 2010
(86) PCT No.: PCT/IL10/00038

§ 371 Date: Feb. 29, 2012

Related U.S. Application Data
(60) Provisional application No. 61/238,434, filed on Aug. 31, 2009.

(51) Int. Cl. A61K 49/10; C07C 69/12; C07C 59/68; C07C 229/36; C07C 211/17; C07C 215/40; C07F 9/06

(52) U.S. Cl. 424/9.3; 564/293; 560/253; 564/15; 562/446; 564/374; 562/478; 564/381

(57) ABSTRACT

The invention relates to a neurochemical agent comprising at least one isotopically labeled carbon atom directly bonded to at least one deuterium atom, uses thereof for the manufacture of a composition for diagnosing and evaluating a condition or disease and kits comprising said agent. The invention further encompasses methods for diagnosing and evaluating a condition or disease in a subject utilizing a composition of the invention.

### FIELD OF THE INVENTION

This invention generally relates to isotopically labeled neurochemical agents, uses thereof for spin hyperpolarized magnetic resonance spectroscopic imaging, and for diagnosing of conditions and disorders, including neurological conditions and disorders.

### BACKGROUND OF THE INVENTION

The following publications are considered relevant for describing the state of the art in the field of the invention:

WO 2007/052,274

U.S. Pat. No. 6,466,814

U.S. Pat. No. 6,574,495

WO 2007/044,867

U.S. Pat. No. 7,102,354

U.S. Pat. No. 6,311,086

U.S. Pat. No. 6,278,893

US2005/0232864

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification relates.

Magnetic resonance imaging and spectroscopy (MRI/MRS) has become an attractive diagnosing technique in the last three decades. Due to its non-invasive features and the fact that it does not involve the exposure of the diagnosed patient to potentially harmful ionizing radiation, MRI has become the leading diagnosing procedure implemented in all fields of medicine.

The underlying principle of MRI and MRS is based on the interaction of atomic nuclei with an external magnetic field. Nuclei with spin quantum number I=½ (such as ¹H, ¹³C, and ¹⁵N) can be oriented in two possible directions: parallel (“spin up”) or anti-parallel (“spin down”) to the external magnetic field. The net magnetization per unit volume, and thus the available nuclear magnetic resonance (NMR) signal, is proportional to the population difference between the two states. If the two populations are equal, their magnetic moments cancel, resulting in zero macroscopic magnetization, and thus no NMR signal. However, under thermal equilibrium conditions, slightly higher energy is associated with the “spin down” direction, and the number of such spins will thus be slightly smaller than the number of spins in the “spin up” state.

An artificial, non-equilibrium distribution of the nuclei can also be created by hyperpolarization NMR techniques for which the spin population differences is increased by several orders of magnitudes compared with the thermal equilibrium conditions. This significantly increases the polarization of the nuclei thereby amplifying the magnetic resonance signal intensity.

The enhancement of the hyperpolarized magnetic resonance signal is limited by the relatively fast decay of the hyperpolarization due to spin-lattice relaxation (termed as T₁ relaxation time). This decay determines the temporal window of ability to detect the hyperpolarized nuclei. Known techniques of enriching the proton positions with deuterium were shown to prolong the T₁ relaxation times of compounds in a manner that is dependant on the compound's conformation in solution. The prolongation of T₁ values is attributed to a decrease in dipolar interaction that a particular nucleus experiences. However, because the dipolar interaction is only one of several relaxation mechanisms that affect the overall T₁ relaxation time, it is not possible to predict the extent of this effect for a particular nucleus in specific molecule within a specific medium (for example in the blood). Moreover, prolongation of T₁ in itself at times does not allow for practical and effective in vivo magnetic resonance detection of a compound or its metabolic fate when administered to a subject, since the sensitivity of detection is limited due to the low natural abundance of ¹³C nuclei, thereby yielding signals which are below the threshold of detection.

There is a need for isotopically labeled compounds capable of being hyperpolarized giving rise to high T₁ relaxation time values and higher sensitivity of detection, thereby enabling practical and useful non-invasive diagnosing techniques of conditions and disorders in the human body.

### SUMMARY OF THE INVENTION

The present invention provides a neurochemical agent comprising at least one isotopically labeled carbon atom directly bonded to at least one deuterium atom.

In a further aspect the invention provides a neurochemical agent comprising an isotopically labeled carbon atom directly bonded to at least one deuterium atom.

The term “neurochemical agent” as used herein is meant to encompass any agent participating in neurological biochemical pathways, in generation and/or degradation of neurotransmitters and in neuro-energetic pathways. Such agents may cross the blood-brain-barrier by passive or active transport, and be taken up and processed by the cellular system of the nervous system. It is noted that neurochemical agents of the invention may also participate in other metabolic events in the blood circulation in other organs, which are not only limited to cellular events within the nervous system, such as for example in pathological processes within and outside the nervous system such as cancer.

In some embodiments of the invention said neurochemical agent is selected from the following non-limiting list consisting of choline, dopamine, L-DOPA, acetylcholine, tyrosine, N-acetylaspartate, creatine, L-arginine, L-citrulline, L-tryptophan, 5-hydroxy-tryptophan, 5-hydroxy-tryptamine (5-HT, serotonin), glutamate, gamma-aminobutyric acid, norepinephrine, epinephrine, vanillylmandelic acid (VMA), homovanillic acid (HVA), 3-O-methyldopamine (3OMD), 3-O-methylnorepinephrine (3OMN), 3-O-methylepinephrine (3OME), dopaquinone, 5-hydroxyindole acetaldehyde (5-HIA), 5-hydroxyindole acetic acid (5-HIAA), melatonin, rivastigmine tartrate, rasagiline (N-propargyl-1-(R) aminoindan, methylphenidate (methyl 2-phenyl-2-(2-piperidyl)acetate), amphetamine (alpha-methyl-phenethylamine), (S)-2-amino-3-(5-hydroxy-1H-indol-3-yl)propenoic acid, 2-amino-2-ene-5-(diaminomethylidene amino)pentanoic acid and 2-amino-5-(diaminomethylidene imino)pentanoic acid, N-acetylcitrulline, argininosuccinate, kynurenic acid (KYNA), 7-chlorokynurenic acid (7-Cl-KYNA), kynurenine, and 4-chlorokynurenine, tacrine, donepezil, metrifonate, fluoxetine, sertraline, paroxetine, fluvoxamine, citalopram, escitalopram, venlafaxine, nefazodone, mirtazapine, bupropion, cianopramine, femoxetine, ifoxetine, milnacipran, oxaprotiline, sibutramine, viqualine, clozapine, fenclonine, dexfenfluramine, chlorpromazine, methamphetamine, prazosin, terazosin, doxazosin, trimazosin, labetalol, medroxalol, tofenacin, trazodone, viloxazine, riluzole or any metabolite or salt thereof.

In other embodiments of the invention said neurochemical agent is selected from group consisting of choline, betaine, acetylcholine, N-acetylaspartate, L-DOPA, dopamine, norepinephrine, epinephrine, homovanillic acid, 3-O-methyldopamine, 3-O-methylnorepinephrine, 3-O-methylepinephrine, dopaquinone, vanillylmandelic acid, 5-hydroxyindole acetaldehyde, 5-Hydroxyindole acetic acid, melatonin, rivastigmine tartrate, rasagiline (N-propargyl-1-(R)aminoindan), amphetamine (alpha-methyl-phenethylamine), methylphenidate (methyl 2-phenyl-2-(2-piperidyl)acetate), (2-hydroxyethenyl)trimethylammonium, (S)-2-amino-3-(5-hydroxy-1H-indol-3-yl)propenoic acid, (S)-2-amino-3-(3,4-dihydroxyphenyl)propenoic acid, L-citrulline, 2-amino-2-ene-5-(diaminomethylidene amino)pentanoic acid and 2-amino-5-(diaminomethylidene imino)pentanoic acid and any methanolites thereof.

A neurochemical agent of the invention comprises at least one isotopically labeled carbon atom which is directly bonded to at least one deuterium atom (commonly marked as D or ²H).

The term “isotopically labeled atom” is meant to encompass an atom in a compound of the invention for which at least one of its nuclei has an atomic mass which is different than the atomic mass of the prevalent naturally abundant isotope of the same atom. Due to different number of neutrons in the nuclei, the atomic mass of a isotopically labeled atoms is different. The total number of neutrons and protons in the nucleus represents its isotopic number.

In some embodiments an isotopically labeled atom is ¹³C (having 7 neutrons and 6 protons in carbon nucleus). In other embodiments an isotopically labeled atom is ²H (having 1 neutron and 1 proton in hydrogen nucleus). In other embodiments an isotopically labeled atom is ¹⁵N (having 8 neutrons and 7 protons in nitrogen nucleus). As will be appreciated by the description below, the isotopic labeling of specific atoms in a compound of the invention is achieved by techniques known to a person skilled in the art of the invention, such as for example synthesizing compounds of the invention from isotopically labeled reactants or isotopically enriching specific nuclei of a neurochemical agent.

When referring to a neurochemical agent comprising at least one isotopically labeled atom, it should be understood to encompass agents having isotopically labeled atoms above the natural abundance of said at least one isotopically labeled atom. Thus, in some embodiments when said isotopically labeled atom is deuterium, said isotopical enrichment of said deuterium in a specific position in a compound of the invention, may be between about 0.015% to about 99.9%. Thus, in other embodiments when said isotopically labeled atom is ¹³C, said isotopical enrichment of said carbon in a specific position in a compound of the invention, may be between about 1.1% to about 99.9%. Thus, in some other embodiments when said isotopically labeled atom is ¹⁵N, said isotopical enrichment of said nitrogen in a specific position in a compound of the invention, may be is between about 0.37% to about 99.9%. Thus, a compound or a composition of the invention may have different degrees of enrichment of isotopically labeled atoms.

In some embodiments of the invention, the neurochemical agent comprises one, two, three, four or more ¹³C atoms each one directly bonded to one, two, three or four ²H atoms.

In some embodiments a neurochemical agent of the invention has T₁ relaxation time values for ¹³C nucleus of between about 5 to about 500 sec.

In other embodiments of the invention, said neurochemical agent further comprises at last one isotopically labeled nitrogen atom. In some embodiments said at least one isotopically labeled nitrogen atom may be directly bonded to said at least one isotopically labeled carbon atom. In other embodiments said at last one isotopically labeled nitrogen atom may be adjacent (on a neighboring atom) to said at least one isotopically labeled carbon atom.

In other embodiments a neurochemical agent of the invention further comprises at least one additional isotopically labeled carbon atom. In some embodiments said at least one additional isotopically labeled carbon atom may be directly bonded to said at least one isotopically labeled carbon atom. In other embodiments said at least one additional isotopically labeled carbon atom may be adjacent to said at least one isotopically labeled carbon atom.

In yet further embodiments of the invention said neurochemical agent further comprises, at least one additional isotopically labeled hydrogen atom. In some embodiments said at least one additional isotopically labeled hydrogen atom may be bonded to at least one adjacent to said at least one isotopically labeled carbon atom.

In another one of its aspects, the invention provides a neurochemical agent selected from the following list:
[1,1,2,2-D₄,2-¹³C]-choline;
[1,1,2,2-D₄,1-¹³C]-choline;
[1,2-D₂,1-¹³C]-choline;
[1,2-D₂,2-¹³C]-choline;
[D₁₃,1-¹³C]-choline;
[D₁₃,2-¹³C]-choline;
[1,2-D₂,2-¹³C, trimethylamine-D₉]-choline;
[1,2-D₂,1-¹³C, trimethylamine-D₉]-choline;
[1,1,2,2-D₄,2-¹³C, ¹⁵N]-choline: HO-CD₂-¹³CD₂-¹⁵N⁺(CH₃)₃
[1,2-D₂,2-¹³C,¹⁵N]-choline: HO—CHD-¹³CHD-¹⁵N⁺(CH₃)₃
[D₁₃,2-¹³C, ¹⁵N]-choline: HO-CD₂-¹³CD₂-¹⁵N⁺(CD₃)₃
[2-¹³C, D₁₁]-betaine: HO—CO-¹³CD₂-N⁺(CD₃)₃;
[2-¹³C,2,2-D₂]-betaine: HO—CO-¹³CD₂-N⁺(CH₃)₃;
[1,2-¹³C₂, D₁₁, ¹⁵N]-betaine: HO—¹³CO-¹³CD₂-¹⁵N⁺(CD₃)₃
[2-¹³C, D₁₁, ¹⁵N]-betaine: HO—CO-¹³CD₂-¹⁵N⁺(CD₃)₃
[2-¹³C,2,2-D₂]-betaine aldehyde: H—CO-¹³CD₂-N⁺(CH₃)₃;
[1-¹³C,2,2-D₂]-betaine aldehyde: H-¹³CO-CD₂N⁺(CH₃)₃;
[1-¹³C, D₁₁]-betaine aldehyde: H-¹³CO-CD₂-N⁺(CD₃)₃;
[1-¹³C, D₁₁,¹⁵N]-betaine aldehyde: H-¹³CO-CD₂-¹⁵N⁺(CD₃)₃;
[1,1,2,2-D₄,2-¹³C]-acetylcholine;
[D₁₃,2-¹³C]-acetylcholine;
[7,7,8-D₃,7-¹³C]-L-tyrosine;
[7,7,8-D₃,8-¹³C]-L-tyrosine;
[D₇,7-¹³C]-tyrosine;
[D₇,8-¹³C]-L-tyrosine;
[7,7,8-D₃,7-¹³C]-L-DOPA;
[7,7,8-D₃,8-¹³C]-L-DOPA;
[2,5,6,7,7,8-D₆,8-¹³C]-DOPA;
[2,5,6,7,7,8-D₆,7-¹³C]-L-DOPA;
[2,5,6,7,7,8-D₆,7,8-¹³C₂, ring-¹³C₆]-DOPA;
[D₇, ¹³C₈]-dopamine;
[D₇,7-¹³C]-dopamine;
[D₇,8-¹³C]-dopamine;
[D₇, ¹³C₆ ring]-dopamine;
[1,2-D₂,1-¹³C]-(2-hydroxyethenyl)trimethylammonium;
[1,2,D₂,2-¹³C]-(2-hydroxyethenyl)trimethylammonium;
[7-D,7-¹³C]S)-2-amino-3-(3,4-dihydroxyphenyl)propenoic acid: 3-HO-,4HO—C₆H₃¹³CDC(NH₂)COOH;
[7-D,8-¹³C]—(S)-2-amino-3-(3,4-dihydroxyphenyl)propenoic acid
3-HO-,4HO—C₆H₃CD¹³C(NH₂)COOH;
[6,5,2,7-D₄,8-¹³C]—(S)-2-amino-3-(3,4-dihydroxyphenyl)propenoic acid
3-HO-,4HO—C₆D₃CD¹³C(NH₂)COOH;
[6,5,2,7-D₄,7-¹³C]—(S)-2-amino-3-(3,4-dihydroxyphenyl)propenoic acid;
[6,5,2,7-D₄, ¹³C₆ ring]-(S)-2-amino-3-(3,4-dihydroxyphenyl)propenoic acid;
[6,6,6-D₃,6-¹³C]-N-acetylaspartate: HOOCCH(NH(CO¹³CD₃))CH₂COOH
[1,2,2-D₃,1-¹³C]-N-acetylaspartate: HOOC¹³CD(NH(COCH₃))CD₂COOH
[2,2-D₂,2-¹³C]-creatine: H₂N⁺C(NH₂)N(CH₃)¹³CD₂CO₂
[2,2,6,6,6-D₅,2,6-¹³C₂-¹⁵N]-creatine: H₂N⁺C(NH₂)¹⁵N(¹³CD₃)¹³CD₂CO₂
[2,2,6,6,6-D₅,2,6-¹³C₂]-creatine: H₂N⁺C(NH₂)N(¹³CD₃)¹³CD₂CO₂
[2,3,3,4,4,5,5-D₇,2-¹³C]-arginine: ⁺NH₂C(NH₂)NHCD₂CD₂CD₂¹³CD(NH₂) CO₂H
[2,3,3,4,4,5,5-D₇,3-¹³C]-arginine: ⁺NH₂C(NH₂)NHCD₂CD₂¹³CD₂CD(NH₂)CO₂H
[2,3,3,4,4,5,5-D₇,4-¹³C]-arginine: ⁺NH₂C(NH₂)NHCD₂¹³CD₂CD₂CD(NH₂) CO₂H
[2,3,3,4,4,5,5-D₇,5-¹³C]-arginine: ⁺NH₂C(NH₂)NH¹³CD₂CD₂CD₂CD(NH₂)CO₂H
[2,3,3,4,4,5,5-D₇,2-¹³C]-citrulline: NH₂CONHCD₂CD₂CD₂¹³CD(NH₂)CO₂H
[2,3,3,4,4,5,5-D₇,3-¹³C]-citrulline: NH₂CONHCD₂CD₂¹³CD₂CD(NH₂)CO₂H
[2,3,3,4,4,5,5-D₇,4-¹³C]-citrulline: NH₂CONHCD₂¹³CD₂CD₂CD(NH₂)CO₂H
[2,3,3,4,4,5,5-D₇,5-¹³C]-citrulline: NH₂CONH¹³CD₂CD₂CD₂CD(NH₂)CO₂H
[9,9,10-D₃,10-¹³C]-L-tryptophan: C₆H₄C(CD₂-¹³CD(NH₂)COOH)CH—NH
[9,10-D₂,10-¹³C]-tryptophan: C₆H₄C(CDH-¹³CD(NH₂)COOH)CH—NH
[9,9,10-D₃,9-¹³C]-L-tryptophan: C₆H₄C(¹³CD₂-CD(NH₂)COOH)CH—NH
[9,9,10-D₃,10-¹³C]-5-hydroxy-tryptophan: 5-OH—C₆H₃C(CD₂-¹³CD(NH₂)COOH)CH—NH
[9,10-D₂,10-¹³C]-5-hydroxy-tryptophan: 5-OH—C₆H₃C(CDH-¹³CD(NH₂)COOH)CH—NH
[9,9,10-D₃,9-¹³C]-5-hydroxy-tryptophan: 5-OH—C₆H₃C(¹³CD₂-CD(NH₂)COOH)CH—NH
[9,9,10,10-D₄,10-¹³C]-serotonin: 5-OH—C₆H₃C(CD₂-¹³CD₂(NH₂)CH—NH
[9,10-D₂,10-¹³C]-serotonin: 5-OH—C₆H₃C(CDH-¹³CDH(NH₂)CH—NH
[9,9,10,10-D₄,9-¹³C]-serotonin: 5-OH—C₆H₃C(¹³CD₂-CD₂(NH₂)CH—NH
[2,2,3,3,4-D₅,2-¹³C]-glutamate: HOOC¹³CD₂CD₂CD(NH₂)COOH
[2,2,3,3,4-D₅,3-¹³C]-glutamate: HOOCCD₂¹³CD₂CD(NH₂)COOH
[2,2,3,3,4-D₅,4-¹³C]-glutamate: HOOC¹³CD₂CD₂¹³CD(NH₂)COOH
[2,2,3,3,4-D₅,5-¹³C]-glutamate: HOOCCD₂CD₂CD(NH₂)¹³COOH
[2,2,3,3,4-D₅,1-¹³C]-glutamate: HOO¹³CCD₂CD₂CD(NH₂)COOH
[2,2,3,3,4,4-D₆,2-¹³C]-gamma-aminobutyric acid: H₂N-CD₂-CD₂-¹³CD₂-COOH
[2,2,3,3,4,4-D₆,3-¹³C]-gamma-aminobutyric acid: H₂N-CD₂-¹³CD₂-CD₂-COOH
[2,2,3,3,4,4-D₆,4-¹³C]-gamma-aminobutyric acid: H₂N-¹³CD₂-CD₂-CD₂-COOH
[5,6,2,7,8,8-D₆, ¹³C₆]-norepinephrine: 3-HO-,4HO—¹³C₆D₃CD(OH)CD₂-NH₂ (phenyl-¹³C₆)
[5,6,2,7,8,8-D₆, ¹³C₆]-epinephrine: 3-HO-,4HO—¹³C₆D₃CD(OH)CD₂-NH(CH₃) (phenyl-¹³C₆)
[9,9,9-D₃,9-¹³C]-epinephrine: 3-HO-,4HO—C₆H₃CH(OH)CH₂—NH(¹³CD₃) (phenyl-¹³C₆)
[5,6,2,7-D₄, ¹³C₆]-VMA: 3-HO-,4HO—¹³C₆D₃CD(OH)CO₂H (phenyl-¹³C₆)
[5,6,2,7-D₄,7-¹³C]-VMA: 3-HO-,4HO—C₆D₃¹³CD(OH)CO₂H
[5,6,2,7,7-D₅, ¹³C₆]-HVA: 3-HO-,4HO—¹³C₆D₃CD₂CO₂H (phenyl-¹³C₆)
[5,6,2,7,7-D₅,7-¹³C]-HVA: 3-HO-,4HO—C₆D₃¹³CD₂CO₂H
[5,6,2,7,7,8,8,9,9,9-D₁₀,¹³C₆]-3OMD: 3-CD₃O-,4HO—¹³C₆D₃CD₂CD₂NH₂ (phenyl-¹³C₆)
[5,6,2,7,7,8,8,9,9,9-D₁₀,9-¹³C]-3OMD: 3-CD₃O-,4HO—¹³C₆D₃CD₂CD₂NH₂ (3-O-methyl-¹³C)
[5,6,2,9,9,9,7,8,8-D₉,9,7-¹³C₂]-3OMN: 3-¹³CD₃O-,4HO—C₆D₃¹³C₃CD(OH)CD₂NH₂
[9,9,9,10,10,10,2,5,6,7,8,8-D₆,9,7,10-¹³C₂]-3OME: 3-CD₃O-,4HO—C₆D₃CD(OH)CD₂NH(CD₃)
[2,5,6,7,7,8-D₆,8-¹³C]-dopaquinone: 30-,40-C₆D₃CD₂¹³CD(NH₂)COOH
[2,5,6,7,7,8-D₆,7-¹³C]-dopaquinone: 30-,40-C₆D₃¹³CD₂CD(NH₂)COOH
[9,9,-D₂,9-¹³C]-5-HIA: 5-OH—C₆H₃C(¹³CD₂CHO)CH—NH
[9,9,-D₂,9-¹³C]-5-HIAA: 5-OH—C₆H₃C(¹³CD₂CO₂H)CH—NH
[13,13,13,9,9,10,10,12,12,12-D₁₀,9,12,13-¹³C]-melatonin: 5-¹³CD₃O—C₆H₃C(¹³CD₂CD₂NHCO¹³CD₃)CH—NH
[13,13,13,9,9,10,10,12,12,12-D₁₀,10,12,13-¹³C]-melatonin: 5-CD₃O—C₆H₃C(CD₂¹³CD₂NHCO¹³CD₃)CH—NH
[9,9,10,10-D₄,9-¹³C]-melatonin: 5-CH₃O—C₆H₃C(¹³CD₂CD₂NHCOCH₃)CH—NH
[9,9,10,10-D₄,10-¹³C]-melatonin: 5-CH₃O—C₆H₃C(CD₂¹³CD₂NHCOCH₃)CH—NH
[1,1,1,2,2-D₅,1-¹³C]-rivastigmine tartrate: (S)—N-Ethyl-D₅,¹³C—N-methyl-3-[1-(dimethylamino)ethyl]-phenyl carbamate
[16,16,16-D₃,16-¹³C₂]-rivastigmine tartrate
(S)—N-Ethyl-N-methyl-3-[1-(dimethylamino-D₃,¹³C)ethyl]-phenyl carbamate
[13,13,13,12-D₄,13-¹³C]-rivastigmine tartrate
(S)—N-Ethyl-N-methyl-3-[1-(dimethylamino)ethyl-D₄,¹³C]-phenyl carbamate
[13,13,13,12-D₄,12-¹³C]-rivastigmine tartrate
(S)—N-Ethyl-N-methyl-3-[1-(dimethylamino)ethyl-D₄,¹³C]-phenyl carbamate
[16,16,16,15,15,15-D₆,15,16-¹³C₂]rivastigmine tartrate
(S)—N-Ethyl-N-methyl-3-[1-(dimethylamino-D₆,¹³C₂)ethyl]-phenyl carbamate
[3,3-D₂,3-¹³C]-rasagiline
(R)—N-(D₂-prop-2-ynyl)-2,3-dihydro-1H-inden-1-amine
[14,14,14-D₃,14-¹³C]-methylphenidate
methyl-[1)₃,¹³C]phenyl(piperidin-2-yl)acetate
[D₁₈,2-¹³C]-methylphenidate
D₁₈-methyl-[¹³C]phenyl(piperidin-2-yl)acetate-¹³C
[D₁₈,14-¹³C]-methylphenidate
D₁₈-methyl-[¹³C]phenyl(piperidin-2-yl)acetate-¹³C
[9,9,9-D₃,9-¹³C]-amphetamine
1-phenylpropan-2-amine,3,3,3-D₃,3-¹³C
[9,9,9,1,2,2-D₆,1-¹³C]-amphetamine
1-phenylpropan-2-amine,1,1,2,3,3,3-D₆,2-¹³C
[9,9,9,1,2,2-D₆,2-¹³C]-amphetamine
1-phenylpropan-2-amine,1,1,2,3,3,3-D₆,1-¹³C
[9,9,9,1,2,2,4,5,6,7,8-D₁₁,1-¹³C]-amphetamine
1-phenylpropan-2-amine,D₁₁,2-¹³C
[9,9,9,1,2,2,4,5,6,7,8-D₁₁,2-¹³C]-amphetamine
1-phenylpropan-2-amine,D₁₁,1-¹³C
[9-D,9-¹³C]—(S)-2-amino-3-(5-hydroxy-1H-indol-3-yl)propenoic acid: 5-OHC₆H₃C(¹³CDC(NH₂)COOH)CHNH
[9-D,10-¹³C]—(S)-2-amino-3-(5-hydroxy-1H-indol-3-yl)propenoic acid: 5-OHC₆H₃C(CD ¹³C(NH₂)COOH)CHNH
[6,4,3,1,9-D₅,10-¹³C]—(S)-2-amino-3-(5-hydroxy-1H-indol-3-yl)propenoic acid: 5-OHC₆D₃C(CD ¹³C(NH₂)COOH)CDNH
[6,4,3,1,9-D₅,8-¹³C]—(S)-2-amino-3-(5-hydroxy-1H-indol-3-yl)propenoic acid: 5-OHC₆D₃ ¹³C(CDC(NH₂)COOH)CDNH
[3,4,4,5,5-D₅,3-¹³C]-2-amino-2-ene-5-(diaminomethylidene amino)pentanoic acid: ⁺NH₂—C(NH₂)NHCD₂CD₂¹³CDC(NH₂)CO₂H
[3,4,4,5,5-D₅,4-¹³C]-2-amino-2-ene-5-(diaminomethylidene amino)pentanoic acid: ⁺NH₂═C(NH₂)NHCD₂¹³CD₂CDC(NH₂)CO₂H
[3,4,4,5,5-D₅,5-¹³C]-2-amino-2-ene-5-(diaminomethylidene amino)pentanoic acid: ⁺NH₂═C(NH₂)NH¹³CD₂CD₂CDC(NH₂)CO₂H
[2,3,3,4,4,5-D₆,2-¹³C]-2-amino-5-(diaminomethylidene imino)pentanoic acid ⁺NH₂C(NH₂)NCDCD₂CD₂¹³CD(NH₂)CO₂H
[2,3,3,4,4,5-D₆,3-¹³C]-2-amino-5-(diaminomethylidene imino)pentanoic acid ⁺NH₂C(NH₂)NCDCD₂¹³CD₂CD(NH₂)CO₂H
[2,3,3,4,4,5-D₆,4-¹³C]-2-amino-5-(diaminomethylidene imino)pentanoic acid ⁺NH₂C(NH₂)NCD¹³CD₂CD₂CD(NH₂)CO₂H
[2,3,3,4,4,5-D₆,4-¹³C]-2-amino-5-(diaminomethylidene imino)pentanoic acid ⁺NH₂C(NH₂)N¹³CDCD₂CD₂CD(NH₂)CO₂H
[2,3,3,4,4,5-D₆,5-¹³C]-2-amino-5-(diaminomethylidene imino)pentanoic acid: ⁺NH₂C(NH₂)N¹³CDCD₂CD₂CD(NH₂)CO₂H

including any metabolite, salt or derivative thereof.

In some embodiments a neurochemical agent of the invention is in a hyperpolarized state.

As noted above in order to acquire an NMR signal of a particular nucleus of a compound there has to be a significant difference between the spin population energy levels of said nucleus. The strength of the NMR signal is linearly dependent on the number of nuclei at the low energy level. The difference between the population of a nucleus at high and low nuclear energy levels is the “polarization” of the nuclei, which is defined as P=CB₀/T, where C is a nucleus specific constant, B₀ is the magnetic field strength, and T is the absolute temperature. Under thermal equilibrium conditions, the polarization is relatively low thereby resulting in a very weak signal under standard clinical MRI scanners (at body temperature of about 37° C. for a magnetic field of 1.5 T, P (for ¹H)≈5×10⁻⁶ ratio and P (for ¹³C)≈×10⁻⁶ ratio).

In order to increase the polarization of a specific nucleus in a compound consequently creating an artificial, non-equilibrium distribution of the spin population of a nucleus, i.e. a “hyperpolarized” state, where the spin population difference is increased by several orders of magnitudes compared with the thermal equilibrium, the hyperpolarized state can be created ex vivo by means of dynamic nuclear polarization (DNP) techniques, such as the Overhauser effect, in combination with a suitable free radical (e.g. TEMPO and its derivatives). Hyperpolarization may also be performed ex-vivo using the Para-hydrogen Induced Polarization technique, and ortho-deuterium induced polarization. Ex-vivo hyperpolarization may also be performed by interaction with a metal complex and reversible interaction with para-hydrogen without hydrogenation of the organic molecule. These techniques have been described in U.S. Pat. No. 6,466,814, U.S. Pat. No. 6,574,495, and U.S. Pat. No. 6,574,496, and in Adams R. W. et al. (Science, 323, 1708-1711, 2009), the contents of which are incorporated herein by reference.

Ex vivo hyperpolarization of a compound of the invention is performed in order to reach a level of polarization sufficient to allow a diagnostically effective contrast enhancement of said agent. In some embodiments, said level of hyperpolarization may be at least about a factor of 2 above the thermal equilibrium polarization level at the magnetic field strength at which the MRI is performed. In some embodiments, said level of hyperpolarization is at least about a factor of 10 above the thermal equilibrium polarization level at the magnetic field strength at which the MRI is performed. In other embodiments, said level of hyperpolarization is at least about a factor of 100 above the thermal equilibrium polarization level at the magnetic field strength at which the MRI is performed. In yet further embodiments, said level of hyperpolarization is a factor of at least about 1000 above the thermal equilibrium polarization level at the magnetic field strength at which the MRI is performed. In other embodiments said level of hyperpolarization is a factor of at least about 10000 above the thermal equilibrium polarization level at the magnetic field strength at which the MRI is performed. In further embodiments said level of hyperpolarization is a factor of at least 100000 above the thermal equilibrium polarization level at the magnetic field strength at which the MRI is performed.

A hyperpolarized neurochemical agent of the invention comprises nuclei capable of emitting magnetic resonance signals in a magnetic field (e.g. nuclei such as ¹³C and ¹⁵N) and capable of exhibiting T₁ relaxation times between about 5 to 500 sec (at standard MRI conditions such as for example at a field strength of 0.01-5 T and a temperature in the range 20-40° C.). In some embodiments, said hyperpolarized neurochemical agent of the invention has T₂ relaxation times of ¹³C nucleus of between about 10 to 10,000 msec.

In other one of its aspects the invention provides a neurochemical agent of the invention for use in the manufacture of a composition for diagnosing and evaluating a condition or disease.

The term “diagnosing and evaluating a condition or disease” is meant to encompass any process of investigating, identifying, recognising and assessing a condition, disease or disorder of the mammalian body (including its brain). A diagnosis according to the present invention using a neurochemical agent of the invention includes, but is not limited to the objective quantitative diagnosis of a condition or disease, prognosis of a condition or disease, genetic predisposition of a subject to have a condition or disease, efficacy of treatment of a therapeutic agent administered to a subject (either continually or intermittently), quantification of neuronal function, diagnosis and evaluation of a psychiatric, neurodegenerative, and neurochemical diseases and disorders, affirmation of a therapeutic agent activity, determination of drug efficacy, strategic planning of the location of deep brain stimulation electrodes and other neurostimulators, characterization of masses, tumors, cysts, blood vessel abnormalities, and internal organ function; quantification of brain, kidney, liver, and other organs' metabolic function; evaluation and determination of the level of anesthesia, comatose states, and the brain regions affected by stroke or trauma and their penumbra, kidney, liver, and muscle function, examination of the action, response or progress of therapy (involving medicinal and non-medicinal treatment) aimed at alleviating or curing psychiatric, neurodegenerative, and neurochemical diseases and disorders, selection of patients for clinical trials to allow for homogenous groups of patients in terms of neuromodulator activity, especially when the clinical trial is carried out in order to evaluate the efficacy of drugs that are aimed at modifying the level of neuromodulators in the brain and monitoring neuromodulator activity in laboratory animals and in pre-clinical trials, especially when the clinical trial is carried out in order to evaluate the efficacy of drugs that are aimed at modifying the levels of neuromodulators in the brain.

In some embodiments said condition or disease is selected from the following non-limiting list: Alzheimer's disease, Parkinson's diseases, depression, brain injury, dementia, mild cognitive impairment, affective disorders, serotonin syndrome (or hyperserotonemia), neuroleptic malignant syndrome, schizophrenia, addiction, atherosclerosis, and cancer (including brain cancer breast cancer, prostate cancer, pancreatic cancer, ovary cancer, lymphoma and kidney cancer).

The invention further provides a use of a neurochemical agent of the invention for the preparation of a composition for diagnosing and evaluating a condition or disease. The invention further provides a use of a neurochemical agent of the invention for diagnosing and evaluating a condition or disease.

In another one of its aspects the invention provides a use of a neurochemical agent comprising an isotopically labeled carbon atom directly bonded to at least one deuterium atom for the manufacture of a composition for diagnosing and evaluating a condition or disease. In a further aspect the invention provides a use of a neurochemical agent comprising an isotopically labeled carbon atom directly bonded to at least one deuterium atom for diagnosing and evaluating a condition or disease.

In some embodiments of the use of a neurochemical agent of the invention, said neurochemical agent is selected from a group consisting of: choline, betaine, acetylcholine, aspartate, N-acetylaspartate, L-DOPA, dopamine, norepinephrine, epinephrine, homovanillic acid (HVA), 3-O-methyldopamine (3OMD), 3-O-methylnorepinephrine (3OMN), 3-O-methylepinephrine (3OME), dopaquinone, vanillylmandelic acid (VMA), 5-hydroxyindole acetaldehyde (5-HIA), 5-Hydroxyindole acetic acid (5-HIAA), melatonin, rivastigmine tartrate, rasagiline (N-propargyl-1-(R)aminoindan), amphetamine (alpha-methyl-phenethylamine), methylphenidate (methyl 2-phenyl-2-(2-piperidyl)acetate), (2-hydroxyethenyl)trimethylammonium, (S)-2-amino-3-(5-hydroxy-1H-indol-3-yl)propenoic acid, (5)-2-amino-3-(3,4-dihydroxyphenyppropenoic acid, L-citrulline, 2-amino-2-ene-5-(diaminomethylidene amino)pentanoic acid, 2-amino-5-(diaminomethylidene imino)pentanoic acid, aspartatic acid, creatine, L-tyrosine, L-tryptophan, 5-hydroxy-tryptophan, 5-hydroxy-tryptamine (5-HT, serotonin), glutamic acid, gamma-aminobutyric acid and L-arginine.

In other embodiments a use according to the invention relates to neurochemical agents selected from the following list:
[1,1,2,2-D₄,2-¹³C]-choline;
[1,1,2,2-D₄,1-¹³C]-choline;
[1,2-D₂,1-¹³C]-choline;
[1,2-D₂,2-¹³C]-choline;
[D₁₃,1-¹³C]-choline;
[D₁₃,2-¹³C]-choline;
[1,2-D₂,2-¹³C, trimethylamine-D₉]-choline;
[1,2-D₂,1-¹³C, trimethylamine-D₉]-choline;
[2-¹³C,2,2,3,3,3-D₅]-betaine;
[2-¹³C,2,2-D₂]-betaine;
[1,1,2,2-D₄,2-¹³C]-acetylcholine;
[7,7,8-D₃,7-¹³C]-L-tyrosine;
[7,7,8-D₃,7-¹³C]-tyrosine;
[7,7,8-D₂,7-¹³C]-L-DOPA;
[7,7,8-D₃,8-¹³C]-L-DOPA;
[2,5,6,7,7,8-D₆,8-¹³C]-L-DOPA;
[2,5,6,7,7,8-D₆,7-¹³C]-L-DOPA;
[2,5,6,7,7,8-D₆,7,8-¹³C₂, ring-¹³C₆]-DOPA;
[5,6,2,7,7,8,8-D₇, ¹³C₆]-dopamine;
[1,2-D₂,1-¹³C]-(2-hydroxyethenyl)trimethylammonium;
[1,2,D₂,2-¹³C]-(2-hydroxyethenyl)trimethylammonium;
[7-D,7-¹³C]—(S)-2-amino-3-(3,4-dihydroxyphenyl)propenoic acid;
[7-D,8-¹³C]—(S)-2-amino-3-(3,4-dihydroxyphenyl)propenoic acid;
[6,5,2,7-D₄,8-¹³C]—(S)-2-amino-3-(3,4-dihydroxyphenyl)propenoic acid;
[6,5,2,7-D₄,7-¹³C]—(S)-2-amino-3-(3,4-dihydroxyphenyl)propenoic acid;
[6,5,2,7-D₄,1-¹³C]—(S)-2-amino-3-(3,4-dihydroxyphenyl)propenoic acid;
[1,2,2-D₃,1-¹³C]-aspartate: HOOC-¹³CD(NH₂)CD₂COOH
[1,2,2-D₃,2-¹³C]-aspartate: HOOC-CD(NH₂)—¹³CD₂COOH
[1,2-D₂,1,2-¹³C]-aspartate: HOOC-¹³CD(NH₂)—¹³CDHCOOH
[6,6,6-D₃,6-¹³C]-N-acetylaspartate: HOOCCH(NH(CO¹³CD₃))CH₂COOH
[1,2,2-D₃,1-¹³C]-N-acetylaspartate: HOOC¹³CD(NH(COCH₃))CD₂COOH
[2,2-D₂,2-¹³C]-creatine: H₂N⁺C(NH₂)N(CH₃)¹³CD₂CO₂
[2,2-D₂,2,6-¹³C₂,6,6,6-D₃,¹⁵N]-creatine: H₂N⁺C(NH₂)¹⁵N(¹³CD₃)¹³CD₂CO₂⁻
[2,2-D₂,2,6-¹³C₂,6,6,6-D₃]-creatine: H₂N⁺C(NH₂)N(¹³CD₃)¹³CD₂*CO₂⁻
[2,3,3,4,4,5,5-D₇,2-¹³C]-arginine: ⁺NH₂C(NH₂)NHCD₂CD₂CD₂¹³CD(NH₂) CO₂H
[2,3,3,4,4,5,5-D₇,3-¹³C]-arginine: ⁺NH₂C(NH₂)NHCD₂CD₂¹³CD₂CD(NH₂) CO₂H
[2,3,3,4,4,5,5-D₇,4-¹³C]-arginine: ⁺NH₂C(NH₂)NHCD₂¹³CD₂CD₂CD(NH₂) CO₂H
[2,3,3,4,4,5,5-D₇,5-¹³C]-arginine: ⁺NH₂C(NH₂)NH¹³CD₂CD₂CD₂CD(NH₂) CO₂H
[2,3,3,4,4,5,5-D₇,2-¹³C]-citrulline: NH₂CONHCD₂CD₂CD₂¹³CD(NH₂)CO₂H
[2,3,3,4,4,5,5-D₇,3-¹³C]-citrulline: NH₂CONHCD₂CD₂¹³CD₂CD(NH₂)CO₂H
[2,3,3,4,4,5,5-D₇,4-¹³C]-citrulline: NH₂CONHCD₂¹³CD₂CD₂CD(NH₂)CO₂H
[2,3,3,4,4,5,5-D₇,5-¹³C]-citrulline: NH₂CONH¹³CD₂CD₂CD₂CD(NH₂)CO₂H
[9,9,10-D₃,10-¹³C]-L-tryptophan: C₆H₄C(CD₂-¹³CD(NH₂)COOH)CH—NH
[9,10-D₂,10-¹³C]-L-tryptophan: C₆H₄C(CDH-¹³CD(NH₂)COOH)CH—NH
[9,9,10-D₃,9-¹³C]-L-tryptophan: C₆H₄C(¹³CD₂-CD(NH₂)COOH)CH—NH
[9,9,10-D₃,10-¹³C]-5-hydroxy-tryptophan: 5-OH—C₆H₃C(CD₂-¹³CD(NH₂)COOH)CH—NH
[9,10-D₂,10-¹³C]-5-hydroxy-tryptophan: 5-OH—C₆H₃C(CDH-¹³CD(NH₂)COOH)CH—NH
[9,9,10-D₃,9-¹³C]-5-hydroxy-tryptophan: 5-OH—C₆H₃C(¹³CD₂-CD(NH₂)COOH)CH—NH
[9,9,10,10-D₄,10-¹³C]-serotonin: 5-OH—C₆H₃C(CD₂-¹³CD₂(NH₂)CH—NH
[9,10-D₂,10-¹³C]-serotonin: 5-OH—C₆H₃C(CDH-¹³CDH(NH₂)CH—NH
[9,9,10,10-D₄,9-¹³C]-serotonin: 5-OH—C₆H₃C(¹³CD₂-CD₂(NH₂)CH—NH
[2,2,3,3,4-D₅,2-¹³C]-glutamate: HOOC¹³CD₂CD₂CD(NH₂)COOH
[2,2,3,3,4-D₅,3-¹³C]-glutamate: HOOCCD₂¹³CD₂CD(NH₂)COOH
[2,2,3,3,4-D₅,4-¹³C]-glutamate: HOOC¹³CD₂CD₂¹³CD(NH₂)COOH
[2,2,3,3,4-D₅,5-¹³C]-glutamate: HOOCCD₂CD₂CD(NH₂)¹³COOH
[2,2,3,3,4-D₅,1-¹³C]-glutamate: HOO¹³CCD₂CD₂CD(NH₂)COOH
[2,2,3,3,4,4-D₆,2-¹³C]-gamma-aminobutyric acid: H₂N-CD₂-CD₂¹³CD₂-COOH
[2,2,3,3,4,4-D₆,3-¹³C]-gamma-aminobutyric acid: H₂N-CD₂-¹³CD₂-CD₂-COOH
[2,2,3,3,4,4-D₆,4-¹³C]-gamma-aminobutyric acid: H₂N-¹³CD₂-CD₂-CD₂-COOH
[5,6,2,7,8,8-D₆, ¹³C₆]-norepinephrine: 3-HO-,4HO—¹³C₆D₃CD(OH)CD₂-NH₂ (phenyl-¹³C₆)
[5,6,2,7,8,8-D₆, ¹³C₆]-epinephrine: 3-HO-,4HO—¹³C₆D₃CD(OH)CD₂-NH(CH₃) (phenyl-¹³C₆)
[9,9,9-D₃,9-¹³C]-epinephrine: 3-HO-,4HO—C₆H₃CH(OH)CH₂—NH(¹³CD₃) (phenyl-¹³C₆)
[5,6,2,7-D₄, ¹³C₆]-VMA: 3-HO-,4HO—¹³C₆D₃CD(OH)CO₂H (phenyl-¹³C₆)
[5,6,2,7-D₄,7-¹³C]-VMA: 3-HO-,4HO—C₆D₃¹³CD(OH)CO₂H
[5,6,2,7,7-D₅, ¹³C₆]-HVA: 3-HO-,4HO—¹³C₆D₃CD₂CO₂H (phenyl-¹³C₆)
[5,6,2,7,7-D₅,7-¹³C]-HVA: 3-HO-,4HO—C₆D₃¹³CD₂CO₂H
[5,6,2,7,7,8,8,9,9,9-D₁₀,¹³C₆]-3OMD: 3-CD₃O-,4HO—¹³C₆D₃CD₂CD₂NH₂ (phenyl-¹³C₆)
[5,6,2,7,7,8,8,9,9,9-D₁₀,9-¹³C]-3OMD: 3-CD₃O-,4HO—¹³C₆D₃CD₂CD₂NH₂ (3-O-methyl-¹³C)
[5,6,2,9,9,9,7,8,8-D₉,9,7-¹³C₂]-3OMN: 3-¹³CD₃O-,4HO—C₆D₃¹³C₃CD(OH)CD₂NH₂
[9,9,9,10,10,10,2,5,6,7,8,8-D₆,9,7,10-¹³C₂]-3OME: 3-CD₃O-,4HO—C₆D₃CD(OH)CD₂NH(CD₃)
[2,5,6,7,7,8-D₆,8-¹³C]-dopaquinone: 30-,40-C₆D₃CD₂¹³CD(NH₂)COOH
[2,5,6,7,7,8-D₆,7-¹³C]-dopaquinone: 30-,40-C₆D₃¹³CD₂CD(NH₂)COOH
[9,9,-D₂,9-¹³C]-5-HIA: 5-OH—C₆H₃C(¹³CD₂CHO)CH—NH
[9,9,-D₂,9-¹³C]-5-HIAA: 5-OH—C₆H₃C(¹³CD₂CO₂H)CH—NH
[13,13,13,9,9,10,10,12,12,12-D₁₀,9,12,13-¹³C]-melatonin: 5-¹³CD₃O—C₆H₃C(¹³CD₂CD₂NHCO¹³CD₃)CH—NH
[13,13,13,9,9,10,10,12,12,12-D₁₀,10,12,13-¹³C]-melatonin: 5-CD₃O—C₆H₃C(CD₂¹³CD₂NHCO¹³CD₃)CH—NH
[9,9,10,10-D₄,9-¹³C]-melatonin: 5-CH₃O—C₆H₃C(¹³CD₂CD₂NHCOCH₃)CH—NH
[9,9,10,10-D₄,10-¹³C]-melatonin: 5-CH₃O—C₆H₃C(CD₂¹³CD₂NHCOCH₃)CH—NH
[1,1,1,2,2-D₅,1-¹³C]-rivastigmine tartrate: (S)—N-Ethyl-D₅,¹³C—N-methyl-3-[1-(dimethylamino)ethyl]-phenyl carbamate
[16,16,16-D₃,16-¹³C]-rivastigmine tartrate
(S)—N-Ethyl-N-methyl-3-[1-(dimethylamino-D₃,¹³C)ethyl]-phenyl carbamate
[13,13,13,12-D₄,13-¹³C]-rivastigmine tartrate
(S)—N-Ethyl-N-methyl-3-[1-(dimethylamino)ethyl-D₄,¹³C]-phenyl carbamate
[13,13,13,12-D₄,12-¹³C]-rivastigmine tartrate
(S)—N-Ethyl-N-methyl-3-[1-(dimethylamino)ethyl-D₄,¹³C]-phenyl carbamate
[16,16,16,15,15,15-D₆,15,16-¹³C₂]-rivastigmine tartrate
(S)—N-Ethyl-N-methyl-3-[1-(dimethylamino-D₆,¹³C₂)ethyl]-phenyl carbamate
[3,3-D₂,3-¹³C]-rasagiline
(R)—N-(D₂-prop-2-ynyl)-2,3-dihydro-1H-inden-1-amine
[14,14,14-D₃,14-¹³C]-methylphenidate
methyl-[D₃,¹³C]phenyl(piperidin-2-yl)acetate
[D₁₈,2-¹³C]-methylphenidate
D₁₈-methyl-[¹³C]phenyl(piperidin-2-yl)acetate-¹³C
[D₁₈,14-¹³C]-methylphenidate
D₁₈-methyl-[¹³C]phenyl(piperidin-2-yl)acetate-¹³C
[9,9,9-D₃,9-¹³C]-amphetamine
1-phenylpropan-2-amine,3,3,3-D₃,3-¹³C
[9,9,9,1,2,2-D₆,1-¹³C]-amphetamine
1-phenylpropan-2-amine,1,1,2,3,3,3-D₆,2-¹³C
[9,9,9,1,2,2-D₆,2-¹³C]-amphetamine
1-phenylpropan-2-amine,1,1,2,3,3,3-D₆,1-¹³C
[9,9,9,1,2,2,4,5,6,7,8-D₁₁,1-¹³C]-amphetamine
1-phenylpropan-2-amine,D₁₁,2-¹³C
[9,9,9,1,2,2,4,5,6,7,8-D₁₁,2-¹³C]-amphetamine
1-phenylpropan-2-amine,D₁₁,1-¹³C
[9-D,9-¹³C]—(S)-2-amino-3-(5-hydroxy-1H-indol-3-yl)propenoic acid:
5-OHC₆H₃C(¹³CDC(NH₂)COOH)CHNH
[9-D,10-¹³C]—(S)-2-amino-3-(5-hydroxy-1H-indol-3-yl)propenoic acid: 5-OHC₆H₃C(CD ¹³C(NH₂)COOH)CHNH
[6,4,3,1,9-D₅,10-¹³C]—(S)-2-amino-3-(5-hydroxy-1H-indol-3-yl)propenoic acid: 5-OHC₆D₃C(CD¹³C(NH₂)COOH)CDNH
[6,4,3,1,9-D₅,8-¹³C]—(S)-2-amino-3-(5-hydroxy-1H-indol-3-yl)propenoic acid: —OHC₆D₃ ¹³C(CDC(NH₂)COOH)CDNH
[3,4,4,5,5-D₅,3-¹³C]-2-amino-2-ene-5-(diaminomethylidene amino)pentanoic acid: ⁺NH₂═C(NH₂)NHCD₂CD₂¹³CDC(NH₂)CO₂H
[3,4,4,5,5-D₅,4-¹³C]-2-amino-2-ene-5-(diaminomethylidene amino)pentanoic acid: ⁺NH₂═C(NH₂)NHCD₂¹³CD₂CDC(NH₂)CO₂H
[3,4,4,5,5-D₅,5-¹³C]-2-amino-2-ene-5-(diaminomethylidene amino)pentanoic acid: ⁺NH₂═C(NH₂)NH¹³CD₂CD₂CDC(NH₂)CO₂H
[2,3,3,4,4,5-D₆,2-¹³C]-2-amino-5-(diaminomethylidene imino)pentanoic acid
NH₂C(NH₂)NCDCD₂CD₂¹³CD(NH₂)CO₂H
[2,3,3,4,4,5-D₆,3-¹³C]-2-amino-5-(diaminomethylidene imino)pentanoic acid
⁺NH₂C(NH₂)NCDCD₂¹³CD₂CD(NH₂)CO₂H
[2,3,3,4,4,5-D₆,4-¹³C]-2-amino-5-(diaminomethylidene imino)pentanoic acid
⁺NH₂C(NH₂)NCD¹³CD₂CD₂CD(NH₂)CO₂H
[2,3,3,4,4,5-D₆,4-¹³C]-2-amino-5-(diaminomethylidene imino)pentanoic acid
⁺NH₂C(NH₂)N¹³CDCD₂CD₂CD(NH₂)CO₂H
[2,3,3,4,4,5-D₆,5-¹³C]-2-amino-5-(diaminomethylidene imino)pentanoic acid ⁺NH₂C(NH₂)N¹³CDCD₂CD₂CD(NH₂)CO₂H

including any metabolite or derivative thereof.

In another aspect the invention provides a method for diagnosing and evaluating a condition or disease in a subject, said method comprising:
hyperpolarizing a neurochemical agent of the invention comprising at least one isotopically labeled carbon atom directly bonded to at least one deuterium atom;
administering to said subject an effective amount of hyperpolarized neurochemical agent;
monitoring said hyperpolarized neurochemical agent or any metabolite thereof;

thereby diagnosing said neurochemical condition or disease.

The term “monitoring” as used herein is meant to encompass the quantitative and/or qualitative detection and observation of a hyperpolarized neurochemical agent of the invention or its metabolic derivatives administered to said subject. Monitoring may be performed by any non-invasive or invasive imaging method, including, but not-limited to magnetic resonance spectroscopy, magnetic resonance imaging, magnetic resonance spectroscopic imaging, and PET.

In one embodiment said monitoring is performed by means of magnetic resonance spectroscopy using a magnetic resonance scanner (an MRI scanner). Magnetic resonance signals obtained may be converted by conventional manipulations into 2-, 3- or 4-dimensional data (spatial and temporal) including metabolic, kinetic, diffusion, relaxation, and physiological data.

In other embodiments, said magnetic resonance spectroscopy is performed using a double tuned ¹³C/D RF coil. Due to possible coupling between deuterium nuclei and ¹³C-nucleus, the signals ¹³C-signals are split, their intensity is diminished and the signal width is broadened. In order to allow visibility of the agent's or its metabolite signals it is sometimes necessary to improve on the line-width of this signal and increase its intensity. This may be achieved by using a double tuned ¹³C/²H RF coil that is capable of performing deuterium decoupling during the ¹³C acquisition. Various coil design possibilities such as a saddle coil, a birdcage coil, a surface coil, or combinations thereof are suitable for this purpose.

For example, at 11.8 T, it was shown that the deuterium decoupling led to a 3 fold enhancement in the signal of the carbon-13 at position 2 of [1,1,2,2-D₄,2-¹³C]-choline chloride in water. In agreement, the splitting of the signal to a 1:2:3:2:1 multiplet was removed and the overall natural linewidth of this signal (of the split signal envelope) was decreased from about 100 Hz to about 5 Hz. fold.

Further improvement in the signal intensity may be provided utilizing the ¹H-¹³C NOE effect achieved by proton irradiation in addition to ²H irradiation, by means of a triple tuned ¹³C/²H/¹H RF coil that is capable of performing both deuterium and proton decoupling prior to and during the ¹³C acquisition. For example, at 11.8 T, proton NOE and decoupling of [1,1,2,2-D₄,2-¹³C]-choline was achieved by proton irradiation prior to and during ¹³C acquisition. This irradiation led to an increase the signal-to-noise ratio of the ¹³C nucleus at position 2 by a factor of two.

In some embodiments, said subject is administered with consecutive doses of said hyperpolarized neurochemical agent.

The invention further provides a composition comprising at least one neurochemical agent of the invention. It is noted that said composition may comprise at least one neurochemical agent of the invention in a mixture with pharmaceutically acceptable auxiliaries, and optionally other therapeutic agents. The auxiliaries must be “acceptable” in the sense of being compatible with the other ingredients of the composition and not deleterious to the recipients thereof.

Compositions administrable to a subject include those suitable for oral, rectal, nasal, topical (including transdermal, buccal, and sublingual), vaginal or parenteral (including subcutaneous, intramuscular, intravenous, and intradermal) administration or administration via an implant. The compositions may be prepared by any method well known in the art of pharmacy. Such methods include the step of bringing in association a neurochemical agent of the invention with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Compositions suitable for oral administration may be presented as discrete dosage units such as pills, tablets, dragees or capsules, or as a powder or granules, or as a solution or suspension. The active ingredient may also be presented as a bolus or paste. The compositions can further be processed into a suppository or enema for rectal administration.

The invention further includes a composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilized) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulizers or insufflators.

The compounds of the invention may be administered in conjunction with other compounds, including, but not limited to: cholinesterase inhibitors (e.g. rivastigmine), monoamine oxidase inhibitors (e.g. rasagiline), acetylcholine precursors (e.g. choline), dopamine precursor (e.g. L-DOPA), selective serotonin reuptake inhibitors (e.g. fluoxetine), psycostimulants (e.g. methylphenidate), and norepinephrine reuptake inhibitors (e.g. atomoxetine).

In other embodiments, said diagnosis and evaluation is performed during or after said subject is administered with at least one therapeutic agent.

In some embodiments said therapeutic agent is selected from the following non-limiting list: cholinesterase inhibitors (e.g. rivastigmine), monoamine oxidase inhibitors (e.g. rasagiline), acetylcholine precursors (e.g. choline), dopamine precursor (e.g. L-DOPA), selective serotonin reuptake inhibitors (e.g. fluoxetine), psycostimulants (e.g. methylphenidate), and norepinephrine reuptake inhibitors (e.g. atomoxetine).

The invention further provides a kit comprising at least one component containing at least one neurochemical agent of the invention comprising at least one isotopically labeled carbon atom directly bonded to at least one deuterium atom, means for administering said at least one agent and instructions for use.

In some embodiments, said kit is for use in diagnosing and evaluating a neurochemical condition or disease.

It is important to note that temporal and spatial distribution of each neurochemical agent of the invention, including its metabolic derivatives, may be quantified by the methods of the invention and may provide markers of a specific brain activity, psychiatric and neurodegenerative diseases or disorders, and therapeutic action and efficacy.

FIG. 1 depicts the metabolic pathway of the neurochemical agent deuterated-choline to acetylcholine. The carbon-13 nuclei at positions 1 and 2 of the choline molecule can serve as indicators of specific metabolism due to the chemical shift difference of these nuclei in choline and its metabolites. Table 1 shows the major chemical shift differences of ¹³C in choline and acetylcholine.

TABLE 1
Chemical shift differences of ¹³C positions
in choline and acetylcholine.

| | choline (ppm) | acetylcholine (ppm) | Δδ (ppm) |
|---|---|---|---|
| C₁ | 56.5 | 59.2 | 2.7 |
| C₂ | 68.3 | 65.4 | 2.9 |

In Table 2, the carbon-13 (natural abundance) T₁ relaxation times and elongation factors due to two deuteration options possibilities are demonstrated.

TABLE 2
Carbon-13 T₁ relaxation times as measured using choline
labeled at specific positions with deuterium (and ¹³C at
natural abundance). Ninety five percent confidence intervals are
given for each measurement (in brackets).

| | | T₁ of C1 (sec) | T₁ of C2 (sec) |
|---|---|---|---|
| A | choline Cl⁻ | 5.2 | 4.9 |
| | | (4.5, 5.9) | (4.4, 5.4) |
| B | [D₁₃]-choline Br⁻ | 32.0 | 32.9 |
| | | (28.3, 35.3) | (30.3, 35.3) |
| C | [1,1,2,2-D₄]-choline Cl⁻ | 29.1 | 34.9 |
| | | (25.1, 33.1) | (26.4, 43.3) |
| | Elongation factor B/A | 6.2 | 6.7 |
| | Elongation factor C/A | 5.6 | 7.1 |

The data in Table 2 were obtained using a series of inversion recovery studies at 11.8 T which were carried out in order to determine the ¹³C T₁ relaxation times of a deuterated choline molecule and of a partially deuterated choline molecule [1,1,2,2-D₄]-choline Cr. The T₁ of the trimethyl amine position in the fully deuterated choline molecule was 28 sec, which represented an approximate 8 fold elongation factor compared to the native choline molecule in which the trimethyl amine positions are protonated. An example of such a study on fully deuterated choline is shown in FIG. 2. Inversion recovery studies were carried out on a 500 MHz scanner (Varian), equipped with a 5 mm double tuned ¹³C/¹H probe. Carbon-13 signals were detected in these molecules at natural abundance. The number of transitions ranged between 300 to 400 per relaxation delay (r) with a total scanning time of 12 to 67 hours due to a relaxation delay of 130 sec. The data were fitted to the standard inversion recovery equation. The possible effect of concentration on T₁ was investigated in a concentration range of 20 mM to 20M, no significant effect of concentration on T₁ was found in this concentration range. Choline Cl⁻ and [1,1,2,2-D₄]-Choline CU were obtained from Sigma-Aldrich (Israel). Choline-D₁₃ Br⁻ (fully deuterated) was obtained from Cambridge Isotope Laboratories (MA, USA).

The resulting carbon-13 T₁ relaxation rate increased by 7 to 8 fold (compared to the protonated molecules), reaching a duration of 33 to 35 seconds at the methylene positions. The increase in T₁ obtained for the choline enables their hyperpolarized signals for a longer period of time after the hyperpolarization process. This feature enables the utilization of the choline molecule for neurometabolic studies because it enables visualization of the nuclei that display a large enough chemical shift to enable spectral resolution between substrate and product, in this case between choline and acetylcholine. For metabolism in cancer this feature is also important because it enables spectral resolution between choline and its metabolic products phosphocholine and betaine.

Indeed the deuteration of choline led to its visibility on hyperpolarized carbon-13 spectroscopy. A single carbon-13 scan of a DNP hyperpolarized D₁₃-choline in a 5 mm NMR tube, at ¹³C natural abundance showed that all of the three carbon types of the fully deuterated choline are visible with a signal to noise ratio of at least 5:1 in a single scan within 20 sec of the end of the polarization process.

The T₁ of position 2 in [1,1,2,2-D₄,2-¹³C]-choline was further investigated at various magnetic field strengths and temperatures. The liquid state T₁ was measured on a DNP-enhanced liquid state sample at 14.1 T at approx. 37° C. (T₁=48±2 sec) and at at higher temperatures of between about 40 to about 50° C. (T₁=56±4 sec).

In addition, thermal equilibrium T₁ measurement was performed at 9.4 T at 37° C. (T₁=41 sec). The low field T₁ of [1,1,2,2-D₄,2-¹³C]-choline was estimated by placing the enhanced sample in the fringe field of an unshielded 14.1 T magnet (n=2). From this experiment it was concluded that the low field T₁ is long (>40 s) and that the T₁ of the deuterated methylene carbon in choline is less affected by field strength.

The synthetic routes for achieving a neurochemical agent comprising at least one isotopically labeled carbon bonded to at least one deuterium atom are well known to a skilled artisan in the field of the invention. Non limiting examples of such isotopical labeling (enrichment) of an example neurological agent such as choline include non-hydrogenation dependent (DNP and metal complex) and hydrogenation dependent (PHIP) sensitivity enhancement methods depicted in FIGS. 3A-3C and 4A-4B and 4D, respectively.

In FIGS. 3A, 3B and 3C the non-hydrogenation dependent (for DNP and metal complex sensitivity enhancement) synthetic process is depicted. In FIGS. 3A and 3B (1-6), choline is synthesized from ethylene glycol labeled with 4 or 6 deuterium nuclei and 1 or 2 carbon-13 nuclei ([D₄, ¹³C]- or [D₄, ¹³C₂]- or [D₆, ¹³C]- or [D₆, ¹³C₂]-ethylene glycol). Ethylene glycol is reacted with dimethyl amine labeled with D₆ with or without enrichment of ¹⁵N, with or without enrichment of ¹³C, under the reaction conditions specified in FIGS. 3A and 3B (120° C. for 3 h). Dimethyl amine hydrochloride is neutralized before use. The following and final step in this reaction is methylation with methyl iodide to form choline. The resulting labeled compounds are depicted. FIG. 3C (1-6) describes the synthesis of choline from 2-bromoethanol labeled with 2 or 4 deuterium nuclei and carbon-13 (e.g. [D₄,1-¹³C]- or [D₄,2-¹³C]- or [D₄, ¹³C₂]-2-bromoethanol) and trimethyl amine (which may be enriched with D₉ or ¹⁵N or ¹³C) under anhydrous ether and trimethyl amine excess. In the case trimethyl amine hydrochloride is used in this synthesis, the compound is neutralized before use. These reactions follow protocols which were described in Marsella, J. A., Homogeneously catalyzed synthesis of (3-amino alcohols and vicinal diamines from Ethylene Glycol and 1,2-propanediol. J. Org. Chem. 1987, 52, 467-468; and Walz, D. E.; Fields, M.; Gibbs, J. A., The synthesis of choline and acetylcholine labeled in the ethylene chain with isotopic carbon. J. Am. Chem. Soc. 1951, 73, 2968.

Condensed trimethylamine (˜8 ml, ˜90 mmol) was reacted with bromoethanol (0.61 ml, 8 mmol) in an acetone/dry ice bath for 1 h and then in an ice bath 1 h. A single product with the 1H and ¹³C NMR signal characteristics of choline was obtained. In some embodiments, the synthesis of choline for the purpose of preparing isotopically stabilized product for hyperpolarization, may be achieved by the use of hydrogenation dependant (PHIP) approach relaying on the keto-enol tautomerization of betaine aldehyde as a precursor of choline, as shown in FIG. 3D.

According to this embodiment, there may be two strategies for the synthesis of such an enol tautomer as a precursor for hyperpolarized choline: 1) hydrogenation of the enol tautomer of betaine aldehyde, which is thermodynamically less stable, by subjecting the equilibrium to conditions that will drive it to the direction of the enol form, and 2) synthesis of a stable enol tautomer of choline where the enol structure is retained by binding of a “protecting” group to the aldehyde's oxygen atom.

FIG. 4A depicts a general strategy of the first approach. First a choline molecule is oxidized and then a carbon-carbon double bond is hydrogenated. FIG. 4B shows two possibilities for oxidation reactions of choline. The oxidized form of choline exists in a keto-enol equilibrium with the enol form being less abundant. Hydrogenation with para-hydrogen or ortho-deuterium takes place on the less abundant enol form (FIG. 4A at reactions conditions that favor the reduction of a carbon-carbon double bond versus a carbonyl bond, for example at ambient pressure and temperature and using a Rhodium catalyst such as (COD)(DPPB)Rh(I) BF₄. The reduction of the enol form drives the equilibrium towards formation of more of the enol form and hydrogentation continues on the enol form. An example of the feasibility of this approach is shown in FIGS. 4B and 4C. First, betaine aldehyde was synthesized. Then, betaine aldehyde was hydrogenated with a hydrogen mixture enriched with para-hydrogen in the presence of a rhodium catalyst to produce hyperpolarized choline signal at approximately 3.6 ppm (FIG. 4C).

Betaine synthesis was carried out according to procedures described by Rhodes, D; Rich, P J; Myers, A C, et al. Determination of betaines by fast-atom-bombardment mass-spectrometry-identification of glycine betaine deficient genotypes of zea-mays. Plant Physiology Volume: 84 Issue: 3 Pages: 781-788 Published: July 1987; and Lehn, J.-M. EP 1 184 359 A1 2002: (dimethylamino)acetaldehyde diethylacetal (2.3 ml, 12.3 mmol, Sigma-Aldrich) was reacted with methyl iodide (0.9 ml, 14.5 mmol) at 70° C. for 5 h. ¹H-NMR showed a single product (A), MS [M⁺]: 176.16 m/z. The product of this reaction, (trimethylamino)acetaldehyde diethylacetal iodide (A) underwent Dowex-1-Cl—. ¹H-NMR showed a single product (B), MS [M+]: 176.16 m/z. (0.5 g 2.36 mmol) of B were reacted with 8 ml 10% HCl at 55° C., over night. ¹H-NMR of the product in water showed the hydrate form of betaine aldehyde as a single product. in DMSO a mixture of the aldehyde and hydrate was observed, MS [M-H₂O⁺]: 120.07 (100%), [M+] 102.11 (20%) m/z.

The second strategy for the synthesis of an enol tautomer as a precursor for hyperpolarized choline involves the synthesis of a stable enol tautomer of choline where the enol structure is retained by binding of a “protecting” group to the aldehyde's oxygen atom. In this way, prior to the hydrogenation reaction, the reactive aldehyde group is protected to avoid interaction with nucleophiles.

FIG. 4D shows an example for the use of a protecting group incorporated to betaine aldehyde. Such a protecting group is designed to leave the molecule upon hydrogenation of the double bond, resulting in the choline molecule. When the hydrogen used for hydrogenation is enriched with either para-hydrogen or ortho-deuterium, the resulting choline possesses an increased spin order that is then transferred to the adjacent carbon-13.

Another exemplary neurochemical agent is L-DOPA. FIG. 5 shows the metabolic pathway of L-DOPA to dopamine. Table 3 shows the major ¹³C chemical shift differences between similar positions in L-DOPA and dopamine.

TABLE 3
The chemical shift differences between similar
carbon positions in L-DOPA and dopamine.

| | L-DOPA | dopamine | |
|---|---|---|---|
| | δ/ppm | δ/ppm | Δδ |
| C1 | 129.08 | 132.09 | 3.01 |
| C2 | 119.52 | 119.41 | 0.11 |
| C3 | 147.08 | 147.17 | 0.09 |
| C4 | 146.44 | 145.96 | 0.48 |
| C5 | 119.93 | 119.46 | 0.47 |
| C6 | 124.77 | 124.04 | 0.73 |
| C7 | 37.71 | 34.96 | 2.75 |
| C8 | 57.09 | 43.75 | 13.34 |
| C9 | 174.90 | — | — |

The chemical shifts shown in Table 3 demonstrate that positions 1, 2, and 3 of L-DOPA and dopamine allow for metabolic resolution between L-DOPA and dopamine.

Inversion recovery studies were carried out on protonated L-DOPA and dopamine and partially deuterated dopamine a in order to determine the ¹³C T₁ relaxation times in these molecules. The inversion recovery studies were carried out on a 500 MHz scanner (Varian), equipped with a 5 mm double tuned ¹³C/¹H probe. Carbon-13 signals were detected in these molecules at natural abundance. The number of transitions ranged between 300 to 400 per relaxation delay (t) with a total scanning time of 12 to 48 hours. Dopamine HCl and L-DOPA were obtained from Sigma-Aldrich (Israel). [1,1,2,2-D₄]-Dopamine HCl, and [D₃-ring, 2,2-D₂]-Dopamine were obtained from Cambridge Isotope Laboratories (MA, USA).

The results of the inversion recovery studies for carbon positions 1, 2, and 3 in the L-DOPA and dopamine molecule are summarized in Table 4. Despite the comparable elongation factor to the choline molecule, the T₁ values of the methylene positions in dopamine remain below 10 sec and therefore partially deuterated dopamine appears to be unsuitable for use as an injectable hyperpolarized contrast agents in itself.

TABLE 4
¹³C T₁ relaxation times

| | | T₁ of C₁ | T₁ of C₂ | T₁ of C₃ |
|---|---|---|---|---|
| | | (sec) | (sec) | (sec) |
| A | dopamine HCl | 1.1 | 1.0 | 6.4 |
| | | (0.6, 1.3) | (0.2, 2.0) | (4.7, 8.1) |
| B | [1,1,2,2-D₄]-dopamine HCl | 8.7 | 9.3 | 6.3 |
| | (equivalent to 7,7,8,8,-D₄]- | (8.2, 9.1) | (8.6, 10.1) | (6.2, 6.4) |
| | dopamine HCl in the current | | | |
| | document) | | | |
| C | [D₃ ring,2,2-D₂]-dopamine | 1.0 | 4.4 | 5.4 |
| | HCl | (0.7, 1.0) | (1.3, 7.4) | (2.2, 8.6) |
| | (equivalent to D₃ ring,7,7- | | | |
| | D₂]-dopamine HCl in the | | | |
| | current document) | | | |
| D | L-DOPA (non deuterated) | 2.3 | 2.3 | 3.9 |
| | | (0.7, 4.0) | (0.6, 4.0) | (2.3, 5.3) |
| | Elongation factor B/A | 7.9 | 9.3 | 1.0 |
| | Elongation factor C/A | 1.0 | 4.4 | 0.8 |

T₁ values of additional deuterated compounds showed different T₁ elongation factor and overall T₁ values, For example:

1) the compound [2,3,3,4,4,5,5-D₇]-arginine: NH₂C(NH)NHCD₂CD₂CD₂CD(NH₂)CO₂H showed a T₁ of carbons at positions 2, 3, 4, and 5 that is not much greater than 8 sec.
2) The compound [D₇]-L-tryptophan: C₆D₄C(CD₂-CD(NH₂)COOH)CH—NH was dissolved in H₂O and 15% D₂O (54.5 mM) and showed a T₁ of position 10 that is close to 13 sec at 11.8 T.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows the metabolic pathway of the neurological agent choline to acetylcholine.

FIG. 2 shows the ¹³C inversion recovery study of fully deuterated choline.

FIGS. 3A-3C show a non-hydrogenation dependent (for DNP and metal complex sensitivity enhancement) synthetic process of choline.

FIG. 3D illustrates the equilibrium between keto-enol tautomers of betaine aldehyde and hydrogenation reaction on the enol tautomer which results in the synthesis of choline.

FIGS. 4A-4D depicts the general hydrogenation dependant (PHIP) labeling of choline via oxidation of choline to betaine aldehyde (FIG. 4A); two possibilities for oxidation reaction of choline (FIG. 4B); the result of a PHIP study on betaine aldehyde (FIG. 4C) which demonstrates the appearance of a hyperpolarized signal at about 3.8 ppm on a proton spectrum at 11.8 T; and strategies for protecting group incorporation to form a stable enol form of choline (FIG. 4D).

FIG. 5 shows the metabolic pathway of L-DOPA to dopamine.

FIG. 6 shows the ¹³C spectra of the head of a male mouse, 14 weeks old, administered with a dose of 30 mg/kg (200 microliter injected volume) of hyperpolarized [1,1,2,2-D₄,2-¹³C]-choline after treatment with atropine (1 mg/kg) and eserine (0.1 mg/kg), 30 min prior to a hyperpolarized choline injection. The spectra were recorded with a high power pulse (maximal signal intensity achieved with this coil).

FIG. 7 shows the ¹³C spectra of the head of a male mouse, 14 weeks old, administered with at a dose of 30 mg/kg (200 microliter injected volume) of hyperpolarized [1,1,2,2-D₄,2-¹³C]-choline after treatment with atropine (1 mg/kg) and eserine (0.1 mg/kg), 30 min prior to a hyperpolarized choline injection. The spectra were recorded with a low power pulse (⅓ of the maximal signal intensity achieved with this coil).

FIG. 8 shows the ¹³C spectra of the head of a male mouse, 8 weeks old, administered with a dose of about 30 mg/kg (about 2.5 ml injected) of hyperpolarized [1,1,2,2-D₄,2-¹³C]-choline treated with atropine (1 mg/kg) and eserine (0.1 mg/kg), 30 min prior to a hyperpolarized choline injection. The spectra were recorded with a low power pulse (⅙ of the maximal signal intensity achieved with this coil).

FIG. 9 shows the ¹³C spectra of the head of a male mouse, 8 weeks old, administered with a dose of 46 mg/kg (about 2.5 ml injected) of hyperpolarized [1,1,2,2-D₄,2-¹³C]-choline treated with atropine (1 mg/kg) and eserine (0.1 mg/kg), 30 min prior to a hyperpolarized choline injection. The spectra were recorded with a low power pulse (⅙ of the maximal signal intensity achieved with this coil).

FIG. 10 shows the ¹³C spectra of the head of a male mouse, 8 weeks old, administered with a dose of 52 mg/kg (about 2.5 ml injected) of hyperpolarized [1,1,2,2-D₄,2-¹³C]-choline treated with atropine (1 mg/kg), 46 min prior to a hyperpolarized choline injection. The first spectrum was recorded with a low power pulse (⅙ of the maximal signal intensity achieved with this coil, the rest of the spectra were recorded with higher power pulse (maximal signal achieved with this coil).

FIG. 11 shows the synthesis of [7,8-D₂]-L-DOPA with protecting groups (BW-33) by hydrogenation of MADP with D₂

FIG. 12 shows a process for the removal of protecting groups from BW-33 molecule.

FIG. 13 shows a reaction with of MADP with D₂ in the presence of a PHIP catalyst.

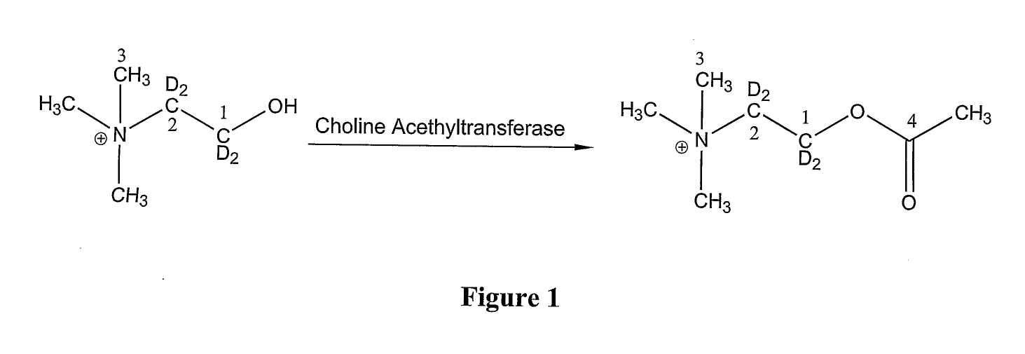

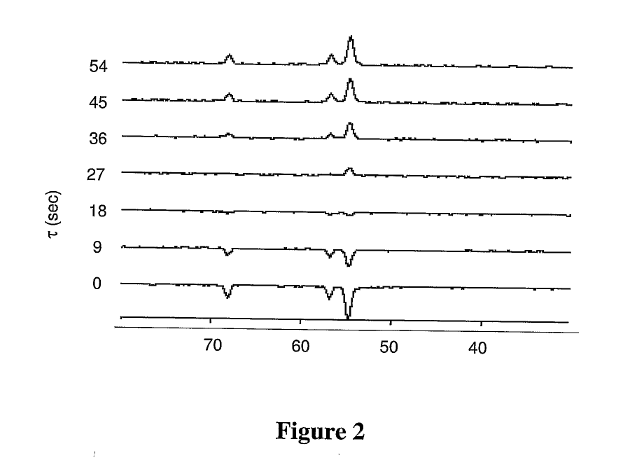

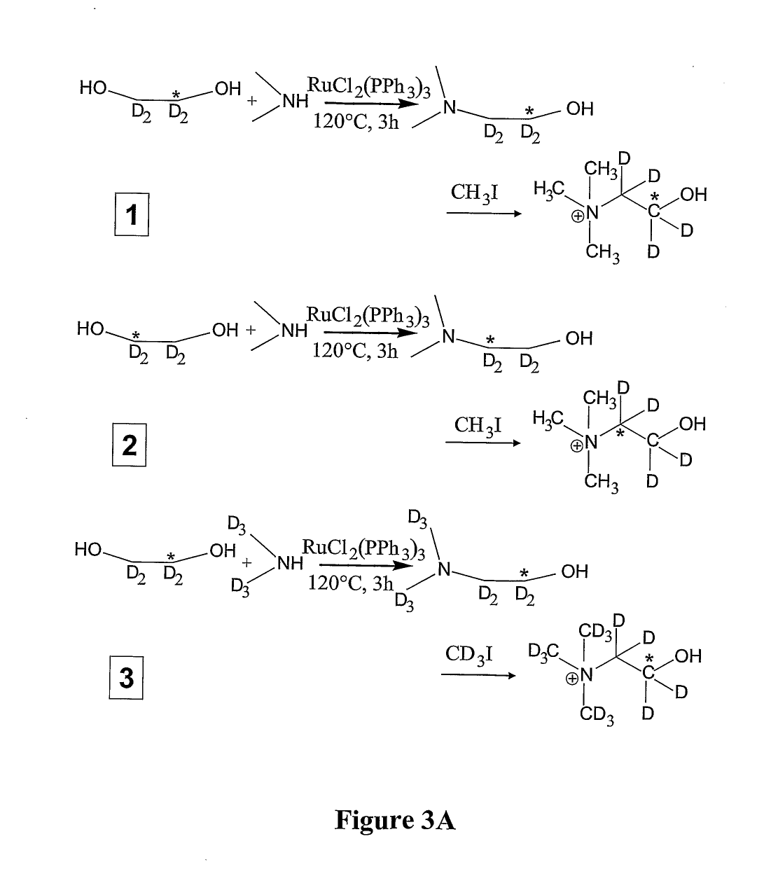

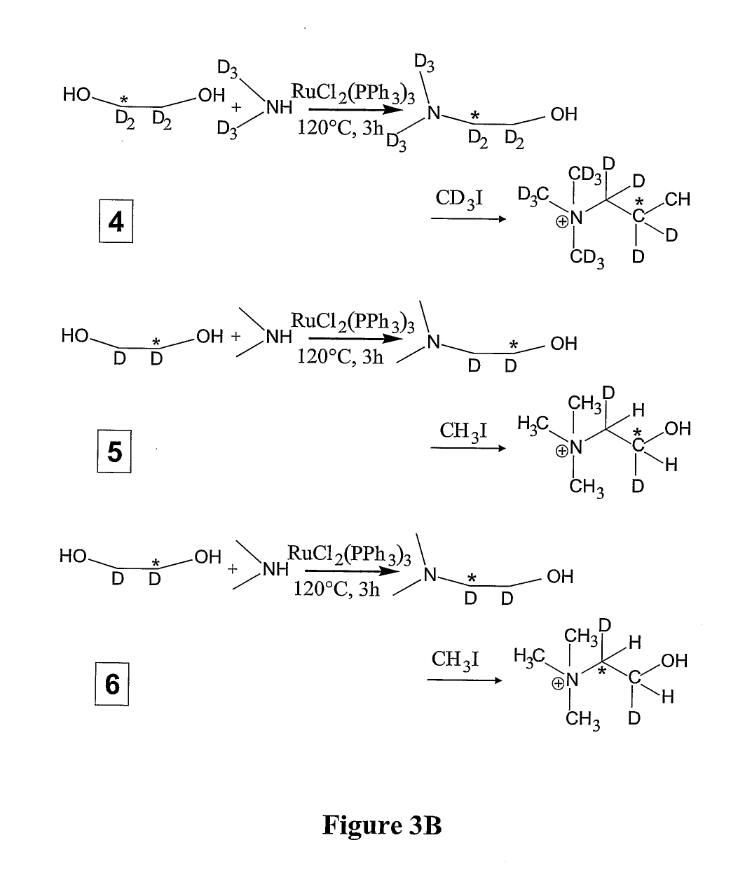

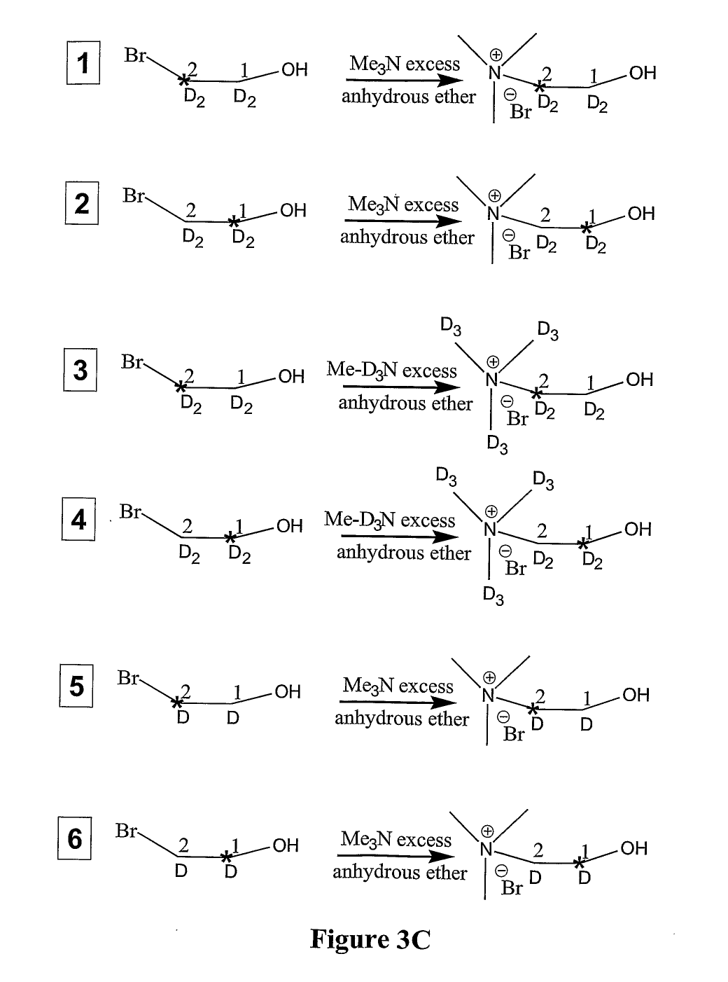

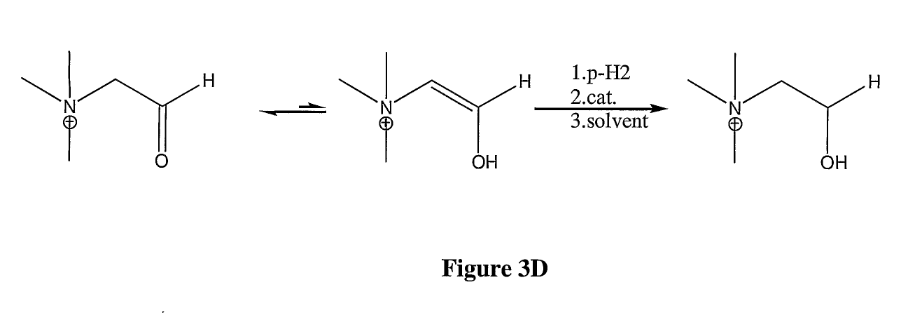

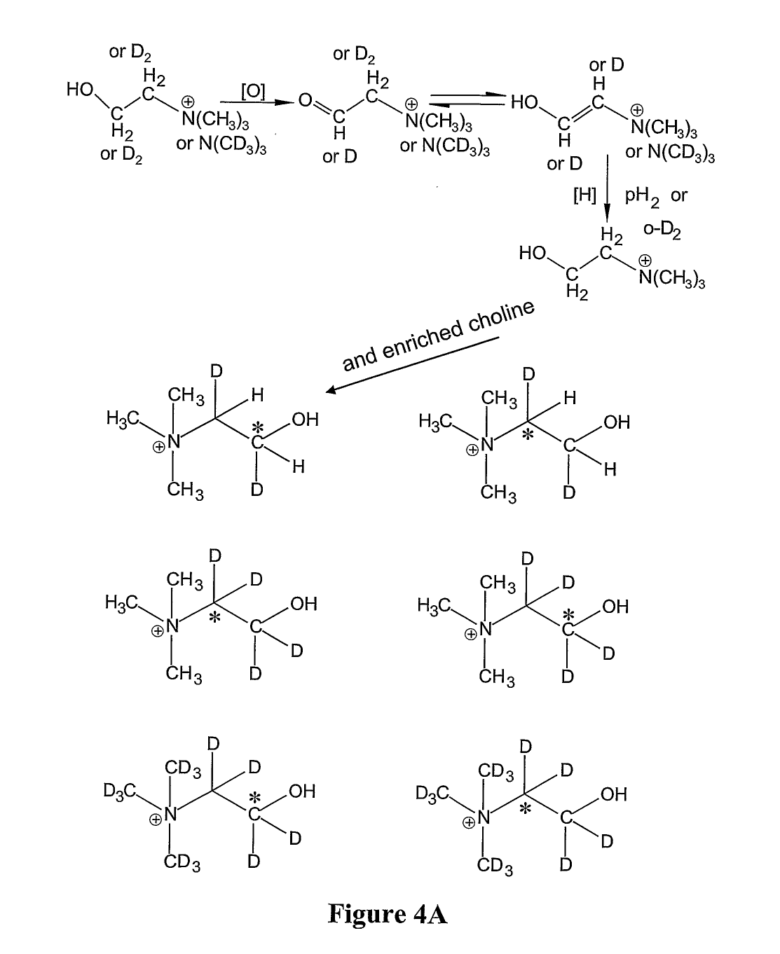

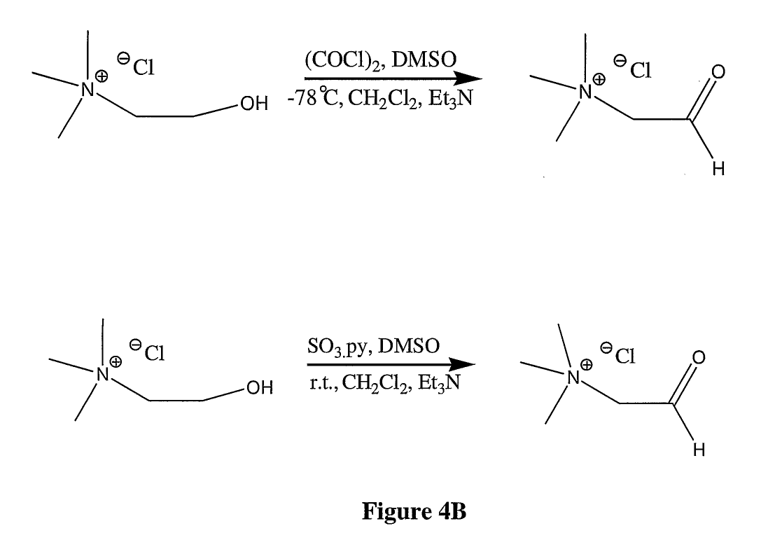

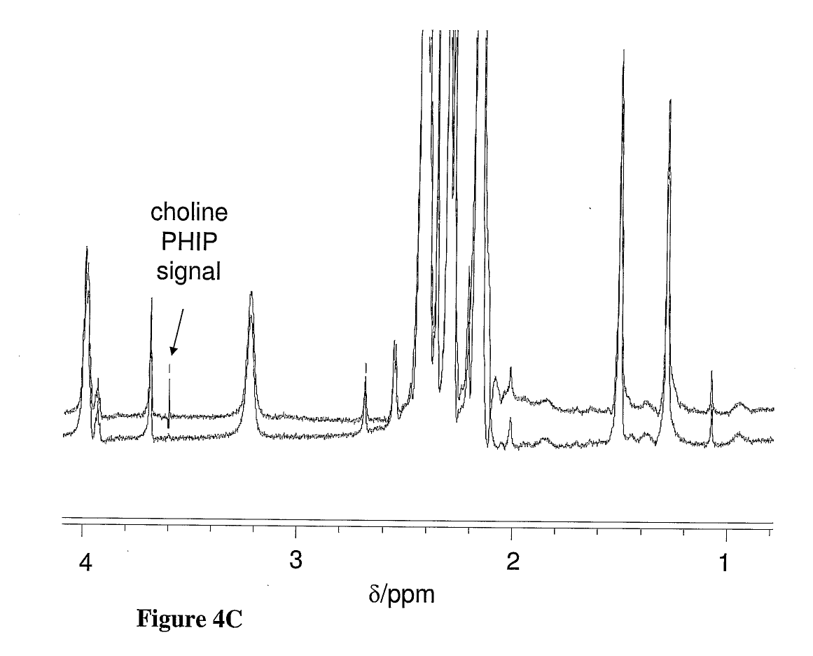

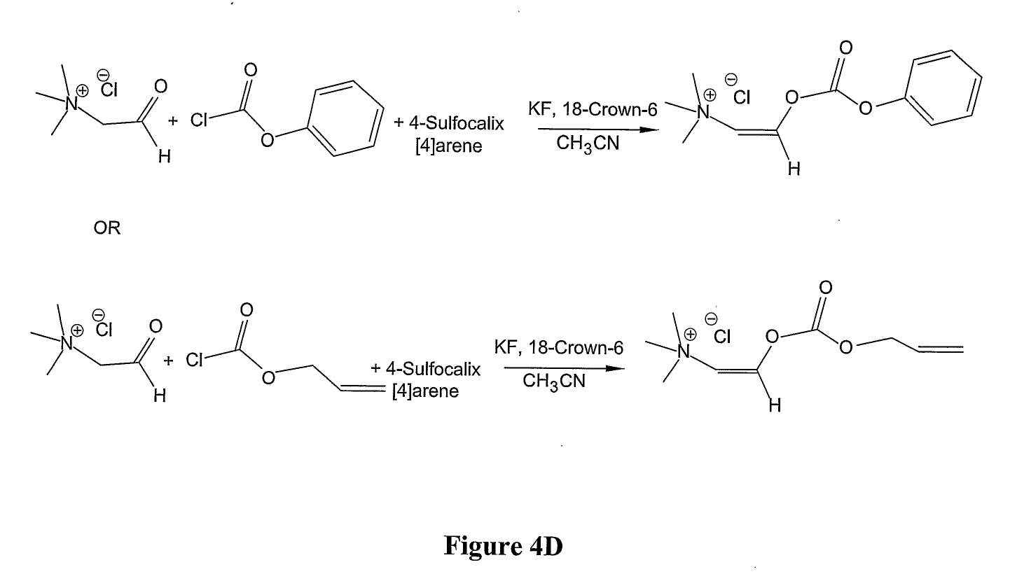

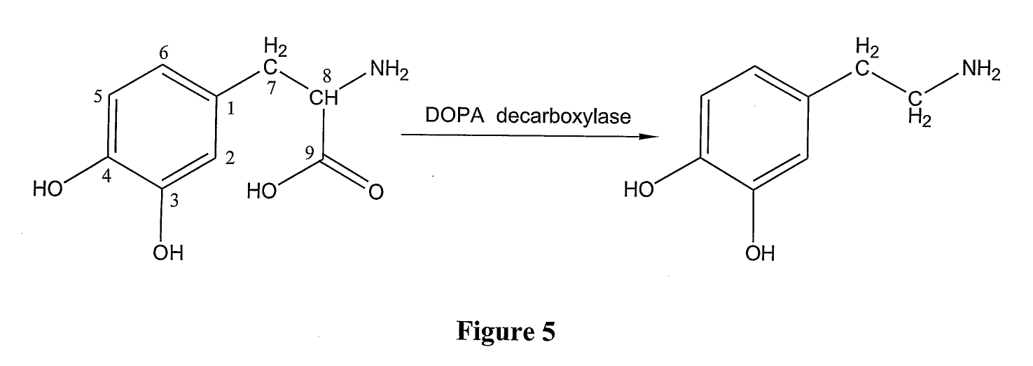

### DETAILED DESCRIPTION OF EMBODIMENTS

The invention is illustrated by the following Examples in a non-limiting manner:

### Example 1

### Acetylcholine Synthesis in the Brain

Optional initial step: The subject is pretreated with atropine prior to choline injection to prevent cholinergic intoxication.

[1,1,2,2-D₄,2-¹³C]-choline is dissolved in 50:50 DMSO:D₂O containing a trityl radical at 1, or 5, or 10, or 15, or 20, or 25 mM. The mixture is placed in an open top chamber.

The mixture is polarized by microwaves for at least one hour at a field of 2.5 T at a temperature of 4.2 K (or lower). According to the previously published procedure (Ardenkjaer-Larsen, J. (2001) U.S. Pat. No. 6,278,893).

When a suitable level of polarization has been reached, the chamber is rapidly removed from the polarizer and, while handled in a magnetic field of no less than 50 mT, the contents are quickly discharged and dissolved in warm saline (40° C., 5 ml).

The solution containing the polarized [1,1,2,2-D₄,2-¹³C]-choline (2, or 3, or 4, 5 ml, or more, the HTNC) is injected to the subject via intravenous catheter that is placed in advance.

The hyperpolarized solution is followed by 20 ml of saline or another routine wash volume.

Experiment 1

Step 1) An anatomic image of the brain is recorded beforehand and the location of the hippocampus is prescribed.

Step 2) One s, or 2 s, or 3 s, or 4 s, or 5 s, or 6 s, or 10 s, or 15 s, or 20 s, or 40 s, or 60 s after injection, a carbon-13 spectrum is recorded from a 1×1×1 cm³ (or 0.5×0.5×0.5 cm³, or 0.2×0.2×0.2 cm³, or 2×2×2 cm³), voxel (single voxel spectroscopy) located at the subject's hippocampus.

Step 3) The spectrum is Fourier transformed and the level of [1,1,2,2-D₄,2-¹³C]-choline and [1,1,2,2-D₄,2-¹³C]-acetylcholine in the subject's hippocampus is quantified. Other potential metabolic products of [1,1,2,2-D₄,2-¹³C]-choline such as [1,1,2,2-D₄,2-¹³C]-betaine, and [1,1,2,2-D₄,2-¹³C]-phosphocholine are quantified as well, simultaneously.

Experiment 2

Experiment 1 is repeated at a different location in the brain, for example the frontal lobe.

Experiment 3

Experiments 1 or 2 performed, with step 2 including a spectroscopic imaging sequence, sampling a slice in the brain at a selected level. The in plane resolution of the spectroscopic image is 0.2 cm, or 0.4 cm, or 0.5 cm, 1 cm, 2 cm, or 3 cm.

The slice thickness is 0.2 cm, or 0.4 cm, or 0.5 cm, or 1 cm, 2 cm, 5 cm, or 10 cm.

Alternatively, a multislice spectroscopic imaging sequence can be applied to sample the entire brain.

Experiment 4

Experiments 1 or 2 or 3 are performed on a group of 3, 5, 10, or 50, or 100 animals (for example, mice, rats, rabbits, mini-pigs, or pigs).

The experiment is repeated on the same group of animals (a few days later) or on a different group of animals, this time while the animals receive a drug that is aimed at modifying the acetylcholine level in the brain, for example, a novel or well-known acetylcholine esterase inhibitor therapy.

The individual and the average rate of choline uptake and acetylcholine synthesis in the normal animal brain are calculated, and drug efficacy is determined.

Alternatively, the experiment is carried out on the group of animals that have been used to develop an animal model of disease, for example a neurodegenerative disease, for example a one sided lesion to the septo-hippocampal pathway, for example a lesion or transection of the fimbria-formix pathway. By comparing between the animals that serve as animal model of disease and normal healthy animals, or by comparing the lesioned side to the control side in a unilateral disease model, the quality, efficacy, and utility of the animal model is assessed and determined.

Experiment 5

Experiments 1 or 2 or 3 or 4 are performed on a group of 3, or 5, or 10, or 50, or 100, or 200, or 500 healthy volunteers who may have no indication of a neurologic or psychiatric disorders and may have no history or current drug addiction or use.

The individual and the average rate of choline uptake and acetylcholine synthesis in the normal human brain are calculated. The maximal level of synthesized acetylcholine is determined as well. The maximal levels of synthesized betaine and phosphocholine are determined as well.

The same experiment is performed in a group of patients who are diagnosed with mild cognitive impairment or various degrees of Alzheimer's disease who are not medicated.

The individual and the average rate of choline uptake and acetylcholine synthesis in the brain within this group of patients as well as the rate of synthesis of betaine and phosphocholine and choline washout rate are calculated. The maximal level of synthesized acetylcholine in these patients is determined as well.

The same experiment is performed in a group of patients who are receiving a novel drug treatment or an existing acetylcholine esterase inhibitor drug treatment (such as rivastigmine).

The individual and the average rate of choline uptake and acetylcholine synthesis in the brain within this group of treated patients are calculated.

By comparison, the drug efficacy in individuals as well as in groups of patients can be determined. Individuals can be monitored routinely at reasonable time durations to confirm continued treatment effectiveness.

Experiment 6

Experiments 1 or 2 or 3 or 4 are performed in the same subject or patient, several times trough the day and night, to determine patterns of choline transport and acetylcholine synthesis. The individual's pattern of acetylcholine synthesis and release is used to design an individualized schedule of controlled acetylcholine release from a controlled release device that is implanted in the subject's brain or a controlled release of choline into the brain or circulation.

Experiment 7

Experiments 1, or 2, or 3, or 4 are performed in a patient that has been diagnosed with a brain tumor. The level and rate of [1,1,2,2-D₄,2-¹³C]-choline transport, [1,1,2,2-D₄,2-¹³C]-phosphocholine synthesis, and [1,1,2,2-D₄,2-¹³C]-betaine synthesis in the investigated tissue aid in the characterization of the tumor or the malignant potential at the tissue surrounding the tumor, as it is known in the art that choline metabolism is altered in malignant tissues.

An extension of this experiment is the characterization of tumors in the body, such as tumors in the breast, prostate, and kidney is possible.

### Example 2

### Dopamine Synthesis in the Brain

[7,7-D₂,8-D,8-¹³C]-L-DOPA (5, or 10, or 15, 20 mg or more) is hyperpolarized and dissolved according to the procedure described in Example 1.

The subject may be pretreated with a single dose or several doses of aromatic-L-amino-acid decarboxylase inhibitor such as carbidopa or benserazide, or difluoromethyldopa, or α-methyldopa (20 mg, 40 mg, 60 mg, or 80 mg) given orally.

1 hour after pretreatment with carbidopa, the hyperpolarized solution (cooled to 37° C. or less), is quickly injected to the subject (preferably in less than 10 sec, or as described in Example 1).

Experiment 1

Step 1) Similar to Example 1, Experiment 1, Step 1.

Step 2) Similarly to Example 1, Experiment 1, Step 2, carbon-13 magnetic resonance spectra are recorded from a single volume element located at a specific location such as the substantia nigra, striatum, basal ganglia, or the thalamus of the subject.

Step 3) The spectra are Fourier transformed and the levels of [7,7-D₂,8-D,8-¹³C]-L-DOPA, [7,7-D₂,8-D,8-¹³C]-dopamine, [7,7-D₂,8-D,8-¹³C]-homovanillic acid, and [7,7-D₂,8-D,8-¹³C]-3-O-methyldopamine and other potential metabolic products of [7,7-D₂,8-D,8-¹³C]-L-DOPA, at the specific location, are quantified, simultaneously.

Experiment 2

Repeated measurements of the types that are described in Experiment 1, and kinetic analysis as described in Example 1, Experiment 2.

Experiment 3

Spectroscopic imaging of the distribution of [7,7-D₂,8-D,8-¹³C]-L-DOPA, [7,7-D₂,8-D,8-¹³C]-dopamine, and other potential metabolites of [7,7-D₂,8-D,8-¹³C]-L-DOPA, as described in Example 1, Experiment 4.

Experiment 4

Experiments 1 or 2 or 3 are performed on a group of 3, or 5, or 10, or 50, or 100 animals (for example, rats, rabbits, mini-pigs, pigs).

The experiment is repeated on the same group of animals (a few days later) or on a different group of animals, this time while the animals receive a drug that is aimed at increasing the dopamine level in the brain, for example, a novel or a well-known monoamine oxidase inhibitor therapy.

The level of [7,7-D₂,8-D,8-¹³C]-dopamine and other [7,7-D₂,8-D,8-¹³C]-L-DOPA metabolites in the brain is determined in both groups of animals. The individual and the average rate of [7,7-D₂,8-D,8-¹³C]-L-DOPA uptake and [7,7-D₂,8-D,8-¹³C]-dopamine synthesis in the naive and treated brain are calculated, and drug efficacy is determined.

Experiment 5

Experiments 1 or 2 or 3 are performed on a group of 3, or 5, or 10, or 50, or 100, or 200, or 500 healthy volunteers who may have no indication of a neurologic or psychiatric disorders and may have no history or current drug addiction or use.

The level of [7,7-D₂,8-D,8-¹³C]-dopamine and other [7,7-D₂,8-D,8-¹³C]-L-DOPA metabolites in the normal human brain is determined. The individual and the average rate of [7,7-D₂,8-D,8-¹³C]-L-DOPA uptake and [7,7-D₂,8-D,8-¹³C]-dopamine synthesis in the normal human brain are calculated.

The same experiment is performed in a group of patients who are diagnosed with Parkinson's disease and who are not medicated.

The level of [7,7-D₂,8-D,8-¹³C]-dopamine and other [7,7-D₂,8-D,8-¹³C]-L-DOPA metabolites in the brain of patients with Parkinson's disease is determined. The individual and the average rate of [7,7-D₂,8-D,8-¹³C]-L-DOPA uptake and [7,7-D₂,8-D,8-¹³C]-dopamine synthesis in the brain within this group of patients are calculated.

The same experiment is performed in a group of patients who are receiving a novel or well-known monoamine oxidase inhibitor drug treatment (such as rasagiline).

The level of [7,7-D₂,8-D,8-¹³C]-dopamine and other [7,7-D₂,8-D,8-¹³C]-L-DOPA metabolites in the treated patients is determined. The individual and the average rate of [7,7-D₂,8-D,8-¹³C]-L-DOPA uptake and [7,7-D₂,8-D,8-¹³C]-dopamine synthesis in the treated patients are calculated.

By comparison, the drug efficacy in individuals as well as in groups of patients can be determined. Individuals can be monitored routinely within reasonable time duration to insure drug effectiveness.

Experiment 6

Experiments 1 or 2 or 3 are performed in the same subject or patient, several times trough the day and night, to determine patterns of L-DOPA uptake and dopamine synthesis in the individual's brain. The data are used to design a schedule of controlled release of L-DOPA, dopamine, or a drug such as monoamine oxidase inhibitor, from a controlled release device that is implanted in the subject's brain or a controlled release of L-DOPA and carbidopa into the circulation.

Alternatively, if deep brain stimulation (DBS) is being considered as a therapeutic route, the data are used to aid in determination of the best location for placing DBS electrodes. After placement of DBS electrodes, similar data may be acquired to determine the effects of DBS on dopamine metabolism in other regions in the brain, for example in the substantia nigra.

Experiment 7

[7-D,8-¹³C]—(S)-2-amino-3-(3,4-dihydroxyphenyl)propenoic acid or [8-¹³C]-(S)-2-amino-3-(3,4-dihydroxyphenyl)propenoic acid (5, 10, 15, or 20 mg or more) is hydrogenated with a hydrogen gas mixture enriched with parahydrogen or ortho-deuterium in the presence of a hydrogenation catalyst or an asymmetric hydrogenation catalyst. The hydrogenation catalyst is separated from the DOPA product using a filtration column, or molecular size sieve, or phase separation (DOPA is more hydrophilic that most catalysts), within a few seconds. Where both D- and L enantiomers of DOPA are produced, they may be quickly separated (in less than 5 sec). The [7,7-D₂,8-D,8-¹³C]-L-DOPA or [7-D,8-D,8-¹³C]-DOPA solution ([D, ¹³C]-labeled-L-DOPA) is undergoing magnetic field cycling to transfer the polarization to the ¹³C nuclei.

The subject is pretreated with a single dose or several doses of aromatic-L-amino-acid decarboxylase inhibitor such as carbidopa or benserazide, or difluoromethyldopa, or α-methyldopa (20 mg, 40 mg, 60 mg, or 80 mg) given orally.

1 hour after pretreatment with carbidopa, the hyperpolarized [D,¹³C]-labeled-L-DOPA_solution (5 ml, the HTNC) is quickly injected to the subject (preferably in less than 10 sec, or as described in Example 1), via intravenous catheter that is placed in advance. The hyperpolarized solution is followed by 20 ml of saline or another routine wash volume.

Experiments 1 through 6 in this example (example 2) are performed. The HTNC is the same in both cases; the difference in experiment 7 is that the hyperpolarization step was achieved via hydrogen induced polarization instead of DNP.

### Example 3

### Dopamine/Acetylcholine Balance in the Brain

The subject is pretreated with atropine and carbidopa as described in Examples 1 and 2.

[7,7-D₂,8-D,8-¹³C]-L-DOPA (5, 10, 15, 20 mg or more) and [1,1,2,2-D₄,2-¹³C]-choline (5, 10, 15, 20 mg or more) are hyperpolarized and dissolved according to the procedure described in Example 1.

The hyperpolarized solution (cooled to 37° C. or less), is quickly injected to the subject (preferably in less than 10 sec, or as described in Example 1).

The solution containing the hyperpolarized [7,7-D₂,8-D,8-¹³C]-L-DOPA and [1,1,2,2-D₄,2-¹³C]-choline (5 ml, the HTNC) is injected to the subject via intravenous catheter that is placed in advance.

The hyperpolarized solution is followed by 20 ml of saline or another routine wash volume.

The balance between acetylcholine production and dopamine production and metabolism is quantified in animal models and in the human brain using the experiments that are described above. Specifically, the effects of existing and novel drugs on this balance is investigated and aids in determination of the drug course of action in situ and drug efficacy.

### Example 4

### In Vivo Carbon-13 Spectroscopy of 1,1,2,2-D₄,2-¹³C-Choline

The compound [1,1,2,2-D₄,2-¹³C]-choline was synthesized and showed the following signals on multinuclei NMR spectra: D-NMR: at c.a. 3.3 ppm—a doublet signal (of 2,2-D₂), at c.a. 3.9 ppm a singlet signal (of 1,1-D₂), at c.a. 4.7 a small signal of natural abundance of HDO in H₂O. ¹³C-NMR: at c.a. 66.8 ppm—a multiplet demonstrating a split signal (of 2-¹³C) due to the close interaction with both 6 deuterons (leading to a split of the signal to five peaks with an intensity ratio of 1:2:3:2:1) and a nitrogen-15 nucleus (leading to a split of the signal to three peaks with a ratio of 1:1:1). ¹H-NMR: at c.a. 3.2 ppm—a singlet signal of the trimethylamine moiety.

In vivo carbon-13 spectroscopy was carried out following injection of hyperpolarized [1,1,2,2-D₄,2-¹³C]-choline to a mouse (n=2). The spectra showed that hyperpolarized [1,1,2,2-D₄,2-¹³C]-choline, and possibly its metabolites as well, were visible for at least 90 seconds from the end of the polarization process. Further studies in rats (n=3) showed similar results and a visible signal more than 3 minutes after the end of the hyperpolarization process (the dissolution).

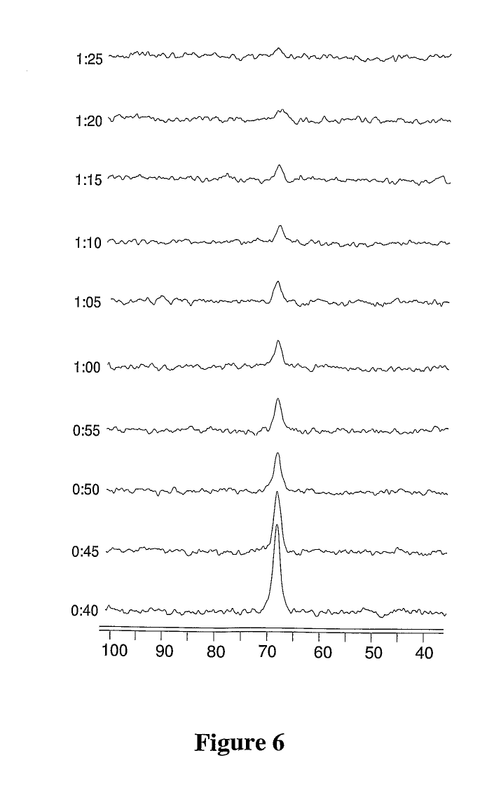

In all of in vitro and in vivo studies, choline was dissolved in 1:1 D₂O:DMSO-d6 solution, a stable free radical was added prior to freezing, and microwave irradiation was performed at about 94.090 GHz. FIGS. 6, 7, 8, 9 and 10 depict the results of these studies.

Experiment 1

A male mouse, 14 weeks old, was treated with atropine (1 mg/kg) and eserine (0.1 mg/kg), 30 min prior to a hyperpolarized [1,1,2,2-D₄,2-¹³C]-choline injection. Hyperpolarized [1,1,2,2-D₄,2-¹³C]-choline was injected at a dose of 30 mg/kg (200 microliter injected volume).

The bolus injection started approximately 20 seconds after the time of dissolution. The bolus duration was about 20 seconds.

¹³C spectra of the rat's head were recorded with an 8 mm ¹³C surface coil every 5 seconds.

As shown in FIG. 6, the first spectrum was recorded 40 seconds after dissolution. The spectra were recorded with a high power pulse (maximal signal intensity achieved with this coil). Exact flip angles are not known due to the inherent B₁ inhomogeneity of a surface coil. The consecutive spectra were processed with exponential multiplication of 30 Hz and phase corrected based on the highest signal (in the first spectrum). Frequency adjustments and zero filling were not applied.

Experiment 2

A male mouse, 14 weeks old, was treated with atropine (1 mg/kg) and eserine (0.1 mg/kg), 30 min prior to a hyperpolarized [1,1,2,2-D₄,2-¹³C]-choline injection. Hyperpolarized [1,1,2,2-D₄,2-¹³C]-choline was injected at a dose of 30 mg/kg (200 microliter injected volume).

The bolus injection started approximately 20 seconds after the time of dissolution. The bolus duration was about 20 seconds.

¹³C spectra of the rat's head were recorded with an 8 mm ¹³C surface coil every 9 seconds.

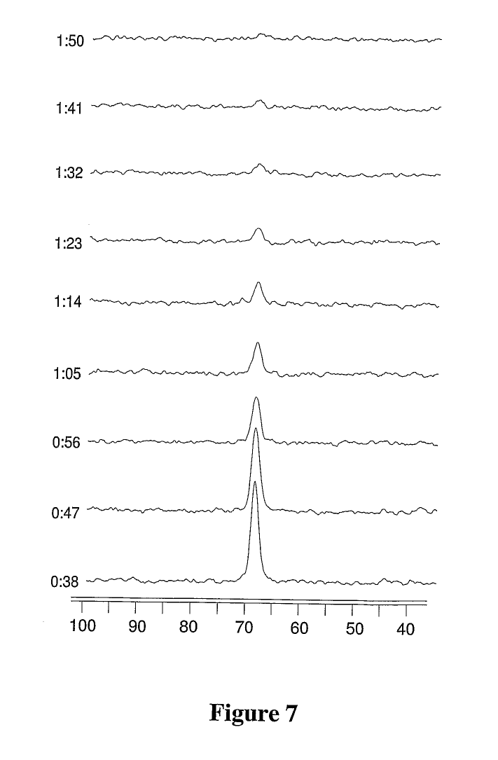

As shown in FIG. 7, the first spectrum was recorded 38 seconds after dissolution. The spectra were recorded with a low power pulse (⅓ of the maximal signal intensity achieved with this coil). Exact flip angles are not known due to the inherent B₁ inhomogeneity of a surface coil. The consecutive spectra were processed with exponential multiplication of 30 Hz and phase corrected based on the highest signal (in the first spectrum). Frequency adjustments and zero filling were not applied.

Experiment 3

A male rat, 8 weeks old, was treated with atropine (1 mg/kg) and eserine (0.1 mg/kg), 30 min prior to a hyperpolarized [1,1,2,2-D₄,2-¹³C]-choline injection. Hyperpolarized [1,1,2,2-D₄,2-¹³C]-choline was injected at a dose of about 30 mg/kg (about 2.5 ml injected).

The bolus injection started 27 seconds after the time of dissolution. The bolus duration was 14 seconds.

¹³C spectra of the rat's head were recorded with an 8 mm ¹³C surface coil every 10 seconds.

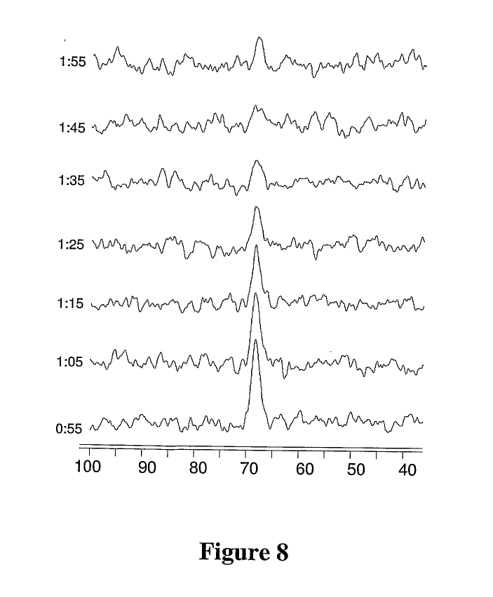

FIG. 8 shows the first spectrum was recorded 55 seconds after dissolution. The spectra were recorded with a low power pulse (⅙ of the maximal signal intensity achieved with this coil). Exact flip angles are not known due to the inherent B₁ inhomogeneity of a surface coil. The consecutive spectra were processed with exponential multiplication of 15 Hz, zero filled to 16384 points, and phase corrected based on the highest signal (in the first spectrum). Frequency adjustments were not applied.

Experiment 4

A male rat, 8 weeks old, was treated with atropine (1 mg/kg) and eserine (0.1 mg/kg), 30 min prior to a hyperpolarized [1,1,2,2-D₄,2-¹³C]-choline injection. Hyperpolarized [1,1,2,2-D₄,2-¹³C]-choline was injected at a dose of about 46 mg/kg (about 2.5 ml injected).

The bolus injection started 22 seconds after the time of dissolution. The bolus duration was 15 seconds.

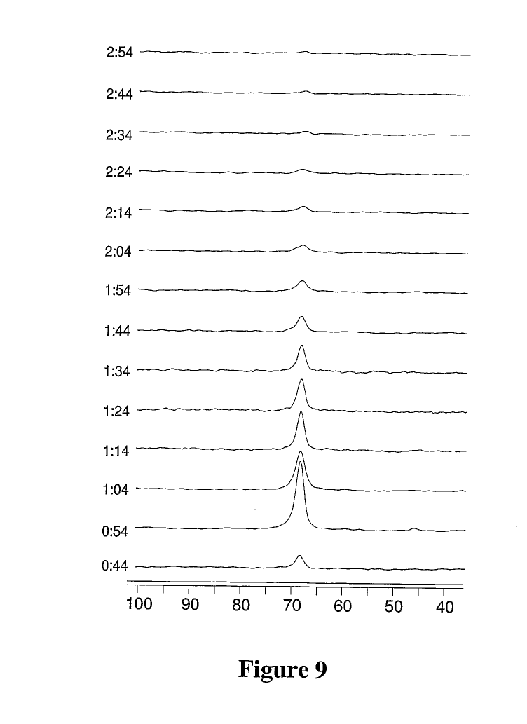

¹³C spectrum of the rat's head was recorded with an 8 mm ¹³C surface coil every 10 seconds, starting at 44 seconds after dissolution (FIG. 9). The spectra were recorded with a low power pulse (⅙ of the maximal signal intensity achieved with this coil). Exact flip angles are not known due to the inherent B₁ inhomogeneity of a surface coil. The consecutive spectra were processed with exponential multiplication of 60 Hz and phase corrected based on the highest signal (in the second spectrum). Frequency adjustments and zero filling were not applied.

Experiment 5

A male rat, 8 weeks old, was treated with atropine (1 mg/kg), 46 min prior to a hyperpolarized [1,1,2,2-D₄,2-¹³C]-choline injection. Hyperpolarized [1,1,2,2-D₄,2-¹³C]-choline was injected at a dose of about 52 mg/kg (about 2.5 ml injected).

The bolus injection started 30 seconds after the time of dissolution. The bolus duration was 13 seconds.

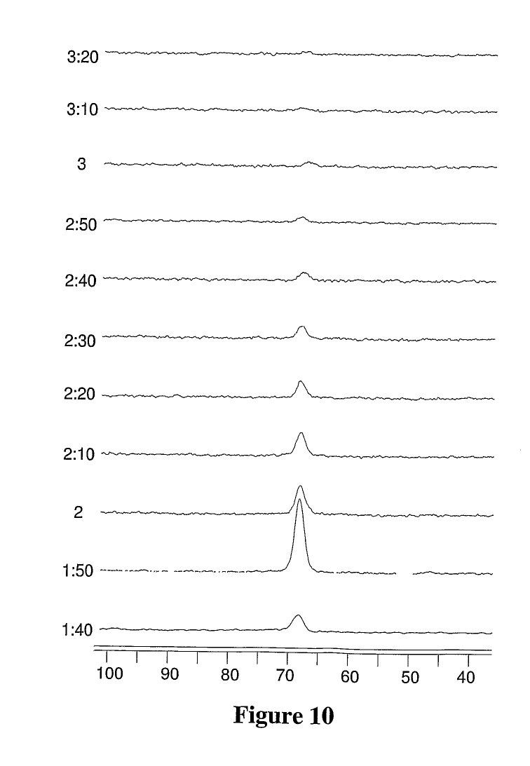

¹³C spectrum of the rat's head (FIG. 10) was recorded with a 15 mm ¹³C surface coil every 10 seconds, starting at 1 minute and 40 seconds after dissolution. The first spectrum was recorded with a low power pulse (⅙ of the maximal signal intensity achieved with this coil, the rest of the spectra were recorded with higher power pulse (maximal signal achieved with this coil). Exact flip angles are not known due to the inherent B₁ inhomogeneity of a surface coil. The consecutive spectra were processed with exponential multiplication of 15 Hz and phase corrected based on the highest signal (in the second spectrum). Frequency adjustments and zero filling were not applied. In this experiment the signal to noise ratio of the hyperpolarized [1,1,2,2-D₄,2-¹³C]-choline in the living rat's head reached a level of about 180:1 almost 2 minutes after the end of the polarization process. This level gradually decayed to about 20:1 ratio, 170 seconds past the end of the polarization process.

### Example 5

### Hyperpolarizing L-DOPA by Hydrogenation Using Enriched Hydrogen and Synthesis of Deuterated L-DOPA

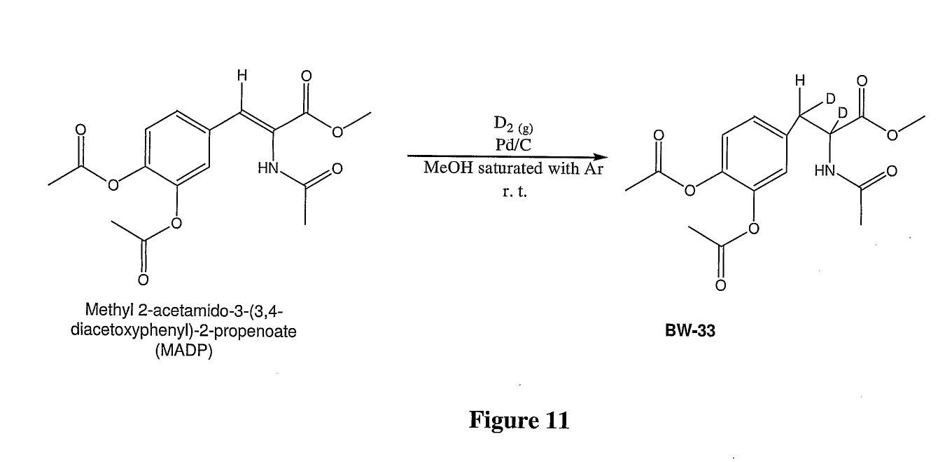

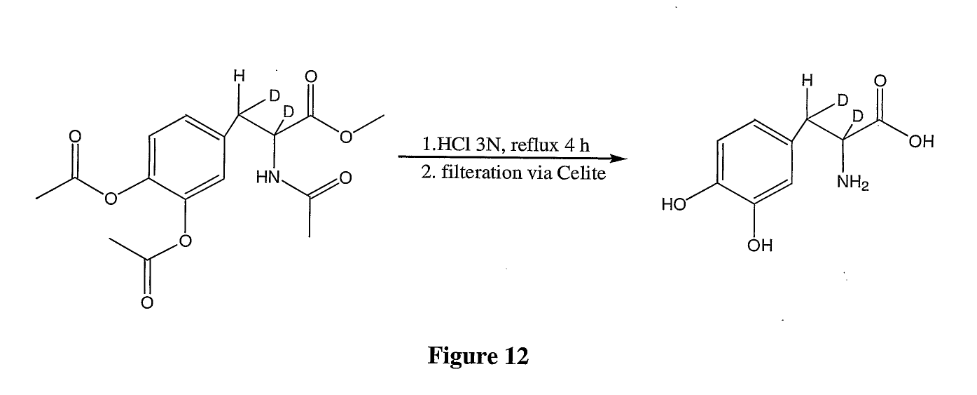

The molecule [7,8-D₂] L-DOPA was synthesized by hydrogenation of methyl 2-acetamido-3-(3,4-diacetoxyphenyl)-2-propenoate (MADP) with D₂ as described in FIG. 11. About 200 ml of D₂ were produced by the interaction of 135 mg NaBD₄ with 3 ml D₂O in the presence of 1% Pt/C for 1 hour and 40 min. MeOH (2.5 ml) was saturated with Ar and combined with 5% Pd/C (11.5 mg) and MADP (97 mg 0.3 mmol), 8 ml of D₂ were consumed by the reaction during 24 h. The protecting groups were removed by acidic reflux as described in FIG. 12. 46 mg of D₂-dihydro-MADP (BW-33) were dissolved in 3 ml 3N HCl, 4 h reflux. 36 mg D₂-L-DOPA for purification was further purified by either Celite filtration or Dowex 50WX4-400 filtration. The D-NMR spectrum of the resulting compound demonstrated the two signals of deuterium at positions 7 and 8 with a 1 ppm difference in chemical shift.

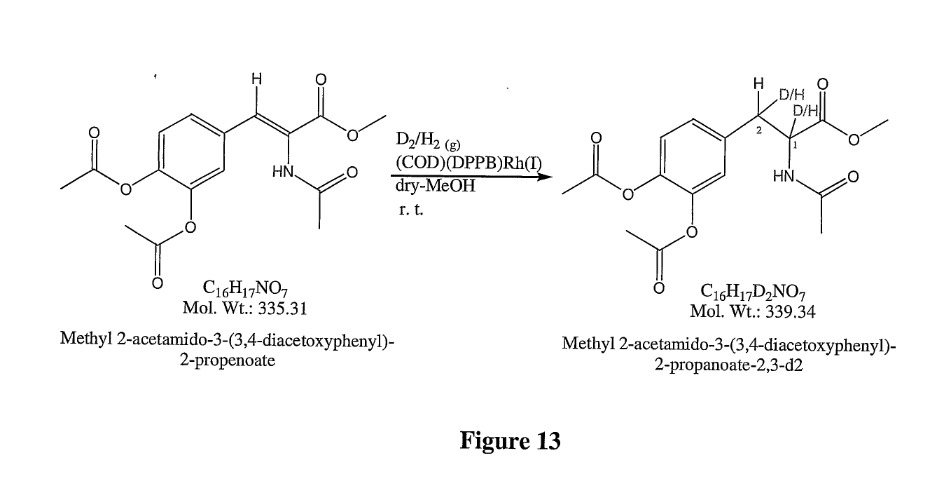

The MADP molecule was investigated also as a precursor for PHIP reactions to yield hyperpolarized L-DOPA. This was carried out by hydrogenation of MADP with either D₂ or H₂ in the presence of a Rhodium catalyst that is suitable for PHIP reactions, as described in FIG. 13.

Experiment 1:

25 mg of MADP was reacted with D₂ (about 1 liter) in the presence of 11 mg Rh catalyst in 700 μl CH₃OH. The deuterium signals at positions 7 and 8 mL-DOPA were identified at approximately 2.05 and 3.15 ppm.

Experiment 2:

25 mg MADP was reacted with H₂ (40 ml) in the presence of 11 mg Rh catalyst in 700 μl CD₃OD where the last 5 ml injected for PHIP effect. When the reaction was performed with an injection of 5 mL hydrogen mixture enriched with para-hydrogen, a small but distinctive anti-symmetric signal was observed at 3.15 ppm. This signal decayed within less than a minute. These results suggested that indeed the MADP molecule can serve as both a para-hydrogen induced polarization (PHIP) and ortho-deuterium induced polarization (ODIP) precursor for the formation of hyperpolarized L-DOPA. More generally, it is shown that the L-DOPA molecule can be hyperpolarized using a precursor that is comprised of a double bond between positions 7 and 8 and protective groups at the sensitive hydroxy/amine/carboxy groups of the molecule. The protective groups selected here for positions 3, 4, and 8 are expected to hydrolase quickly in the blood, in the case that the hydrogenated MADP is injected to an animal or human subject due to the activity of blood esterase enzymes. The protective group at the amine position can be removed by acidic conditions. Therefore, more generally, the potential utility of the PHIP or OCIP approach for hyperpolarization of L-DOPA is shown using a precursor that is comprised of a double bond between positions 7 and 8 and protective groups that hydrolyze quickly when injected to the blood circulation.

1-31. (canceled)

32. A neurochemical agent comprising at least one isotopically labeled carbon atom directly bonded to at least one deuterium atom.

33. A neurochemical agent according to claim 32, comprising an isotopically labeled carbon atom directly bonded to at least one deuterium atom.

34. A neurochemical agent according to claim 32, wherein said isotopically labeled carbon atom is ¹³C.

35. A neurochemical agent according to claim 32, having T₁ relaxation time values of ¹³C nucleus of between about 5 to 500 sec.

36. A neurochemical agent according to claim 32, selected from a group consisting of choline, betaine, acetylcholine, N-acetylaspartate, L-DOPA, dopamine, norepinephrine, epinephrine, homovanillic acid, 3-O-methyldopamine, 3-O-methylnorepinephrine, 3-O-methylepinephrine, dopaquinone, vanillylmandelic acid, 5-hydroxyindole acetaldehyde, 5-Hydroxyindole acetic acid, melatonin, rivastigmine tartrate, rasagiline (N-propargyl-1-(R)aminoindan), amphetamine (alpha-methyl-phenethylamine), methylphenidate (methyl 2-phenyl-2-(2-piperidyl)acetate), (2-hydroxyethenyl)trimethylammonium, (S)-2-amino-3-(5-hydroxy-1H-indol-3-yl)propenoic acid, (S)-2-amino-3-(3,4-dihydroxyphenyl)propenoic acid, L-citrulline, 2-amino-2-ene-5-(diaminomethylidene amino)pentanoic acid and 2-amino-5-(diaminomethylidene imino)pentanoic acid.

37. A neurochemical agent according to claim 32, further comprising at least one isotopically labeled nitrogen atom.

38. A neurochemical agent according to claim 32, further comprising at least one isotopically labeled hydrogen atom.

39. A neurochemical agent according to claim 32, further comprising at least one isotopically labeled carbon atom.

40. A neurochemical agent according to claim 32, selected from the following list:
[1,1,2,2-D₄,2-¹³C]-choline;
[1,1,2,2-D₄,1-¹³C]-choline;
[1,2-D₂,1-¹³C]-choline;
[1,2-D₂,2-¹³C]-choline;
[D₁₃,1-¹³C]-choline;
[D₁₃,2-¹³C]-choline;
[1,2-D₂,2-¹³C, trimethylamine-D₉]-choline;
[1,2-D₂,1-¹³C, trimethylamine-D₉]-choline;
[2-¹³C,2,2,3,3,3-D₅]-betaine;
[2-¹³C,2,2-D₂]-betaine;
[1,1,2,2-D₄,2-¹³C]-acetylcholine;
[7,7,8-D₃,7-¹³C]-L-tyrosine;
[7,7,8-D₃,7-¹³C]-L-tyrosine;
[7,7,8-D₃,7-¹³C]-L-DOPA;
[7,7,8-D₃,8-¹³C]-L-DOPA;
[2,5,6,7,7,8-D₆,8-¹³C]-L-DOPA;
[2,5,6,7,7,8-D₆,7-¹³C]-L-DOPA;
[2,5,6,7,7,8-D₆,7,8-¹³C₂, ring-¹³C₆]-L-DOPA;
[5,6,2,7,7,8,8-D₇, ¹³C₆]-dopamine;
[1,2-D₂,1-¹³C]-(2-hydroxyethenyl)trimethylammonium;
[1,2,D₂,2-¹³C]-(2-hydroxyethenyl)-trimethylammonium;
[7-D,7-¹³C]—(S)-2-amino-3-(3,4-dihydroxyphenyl)propenoic acid;
[7-D,8-¹³C]—(S)-2-amino-3-(3,4-dihydroxyphenyl)propenoic acid;
[6,5,2,7-D₄,8-¹³C]—(S)-2-amino-3-(3,4-dihydroxyphenyl)propenoic acid;
[6,5,2,7-D₄,7-¹³C]—(S)-2-amino-3-(3,4-dihydroxyphenyl)propenoic acid;
[6,5,2,7-D₄,1-¹³C]—(S)-2-amino-3-(3,4-dihydroxyphenyl)propenoic acid;
[6,6,6-D₃,6-¹³C]-N-acetylaspartate: HOOCCH(NH(CO¹³CD₃))CH₂COOH;
[1,2,2-D₃,1-¹³C]-N-acetylaspartate: HOOC¹³CD(NH(COCH₃))CD₂COOH;
[2,2-D₂,2-¹³C]-creatine: H₂N⁺C(NH₂)N(CH₃)¹³CD₂CO₂⁻;
[2,2-D₂,2,6-¹³C₂,6,6,6-D₃,¹⁵N]-creatine: H₂N⁺C(NH₂)¹⁵N(¹³CD₃)¹³CD₂CO₂⁻;
[2,2-D₂,2,6-¹³C₂,6,6,6-D₃]-creatine: H₂N⁺C(NH₂)N(¹³CD₃)¹³CD₂*CO₂⁻;
[2,3,3,4,4,5,5-D₇,2-¹³C]-arginine: ⁺NH₂C(NH₂)NHCD₂CD₂CD₂¹³CD(NH₂)CO₂H;
[2,3,3,4,4,5,5-D₇,3-¹³C]-arginine: ⁺NH₂C(NH₂)NHCD₂CD₂¹³CD₂CD(NH₂)CO₂H;
[2,3,3,4,4,5,5-D₇,4-¹³C]-arginine: ⁺NH₂C(NH₂)NHCD₂¹³CD₂CD₂CD(NH₂)CO₂H;
[2,3,3,4,4,5,5-D₇,5-¹³C]-arginine: ⁺NH₂C(NH₂)NH¹³CD₂CD₂CD₂CD(NH₂)CO₂H
[2,3,3,4,4,5,5-D₇,2-¹³C]-citrulline: NH₂CONHCD₂CD₂CD₂¹³CD(NH₂)CO₂H
[2,3,3,4,4,5,5-D₇,3-¹³C]-citrulline: NH₂CONHCD₂CD₂¹³CD₂CD(NH₂)CO₂H
[2,3,3,4,4,5,5-D₇,4-¹³C]-citrulline: NH₂CONHCD₂¹³CD₂CD₂CD(NH₂)CO₂H
[2,3,3,4,4,5,5-D₇,5-¹³C]-citrulline: NH₂CONH¹³CD₂CD₂CD₂CD(NH₂)CO₂H
[9,9,10-D₃,10-¹³C]-L-tryptophan: C₆H₄C(CD₂-¹³CD(NH₂)COOH)CH—NH
[9,10-D₂,10-¹³C]-L-tryptophan: C₆H₄C(CDH-¹³CD(NH₂)COOH)CH—NH
[9,9,10-D₃,9-¹³C]-L-tryptophan: C₆H₄C(¹³CD₂-CD(NH₂)COOH)CH—NH
[9,9,10-D₃,10-¹³C]-5-hydroxy-tryptophan: 5-OH—C₆H₃C(CD₂-¹³CD(NH₂)COOH)CH—NH
[9,10-D₂,10-¹³C]-5-hydroxy-tryptophan: 5-OH—C₆H₃C(CDH-¹³CD(NH₂)COOH)CH—NH
[9,9,10-D₃,9-¹³C]-5-hydroxy-tryptophan: 5-OH—C₆H₃C(¹³CD₂-CD(NH₂)COOH)CH—NH
[9,9,10,10-D₄,10-¹³C]-serotonin: 5-OH—C₆H₃C(CD₂-¹³CD₂(NH₂)CH—NH
[9,10-D₂,10-¹³C]-serotonin: 5-OH—C₆H₃C(CDH-¹³CDH(NH₂)CH—NH
[9,9,10,10-D₄,9-¹³C]-serotonin: 5-OH—C₆H₃C(¹³CD₂-CD₂(NH₂)CH—NH
[2,2,3,3,4-D₅,2-¹³C]-glutamate: HOOC¹³CD₂CD₂CD(NH₂)COOH
[2,2,3,3,4-D₅,3-¹³C]-glutamate: HOOCCD₂¹³CD₂CD(NH₂)COOH
[2,2,3,3,4-D₅,4-¹³C]-glutamate: HOOC¹³CD₂CD₂¹³CD(NH₂)COOH
[2,2,3,3,4-D₅,5-¹³C]-glutamate: HOOCCD₂CD₂CD(NH₂)¹³COOH
[2,2,3,3,4-D₅,1-¹³C]-glutamate: HOO¹³CCD₂CD₂CD(NH₂)COOH
[2,2,3,3,4,4-D₆,2-¹³C]-gamma-aminobutyric acid: H₂N-CD₂-CD₂¹³CD₂-COOH
[2,2,3,3,4,4-D₆,3-¹³C]-gamma-aminobutyric acid: H₂N-CD₂-¹³CD₂-CD₂-COOH
[2,2,3,3,4,4-D₆,4-¹³C]-gamma-aminobutyric acid: H₂N-¹³CD₂-CD₂-CD₂-COOH
[5,6,2,7,8,8-D₆, ¹³C₆]-norepinephrine: 3-HO-,4HO—¹³C₆D₃CD(OH)CD₂-NH₂ (phenyl-¹³C₆)
[5,6,2,7,8,8-D₆, ¹³C₆]-epinephrine: 3-HO-,4HO—¹³C₆D₃CD(OH)CD₂-NH(CH₃) (phenyl-¹³C₆)
[9,9,9-D₃,9-¹³C]-epinephrine: 3-HO-,4HO—C₆H₃CH(OH)CH₂—NH(¹³CD₃) (phenyl-¹³C₆)
[5,6,2,7-D₄, ¹³C₆]-VMA: 3-HO-,4HO—¹³C₆D₃CD(OH)CO₂H (phenyl-¹³C₆)
[5,6,2,7-D₄,7-¹³C]-VMA: 3-HO-,4HO—C₆D₃¹³CD(OH)CO₂H
[5,6,2,7,7-D₅, ¹³C₆]-HVA: 3-HO-,4HO—¹³C₆D₃CD₂CO₂H (phenyl-¹³C₆)
[5,6,2,7,7-D₅,7-¹³C]-HVA: 3-HO-,4HO—C₆D₃¹³CD₂CO₂H
[5,6,2,7,7,8,8,9,9,9-D₁₀, ¹³C₆]-3OMD: 3-CD₃O-,4HO—¹³C₆D₃CD₂CD₂NH₂ (phenyl-¹³C₆)
[5,6,2,7,7,8,8,9,9,9-D₁₀,9-¹³C]-3OMD: 3-CD₃O-,4HO—¹³C₆D₃CD₂CD₂NH₂ (3-O-methyl-¹³C)
[5,6,2,9,9,9,7,8,8-D₉,9,7-¹³C₂]-3OMN: 3-¹³CD₃O-,4HO—C₆D₃¹³C₃CD(OH)CD₂NH₂
[9,9,9,10,10,10,2,5,6,7,8,8-D₆,9,7,10-¹³C₂]-3OME: 3-CD₃O-,4HO—C₆D₃CD(OH)CD₂NH(CD₃)
[2,5,6,7,7,8-D₆,8-¹³C]-dopaquinone: 30-,40-C₆D₃CD₂¹³CD(NH₂)COOH
[2,5,6,7,7,8-D₆,7-¹³C]-dopaquinone: 30-,40-C₆D₃¹³CD₂CD(NH₂)COOH
[9,9,-D₂,9-¹³C]-5-HIA: 5-OH—C₆H₃C(¹³CD₂CHO)CH—NH
[9,9,-D₂,9-¹³C]-5-HIAA: 5-OH—C₆H₃C(¹³CD₂CO₂H)CH—NH
[13,13,13,9,9,10,10,12,12,12-D₁₀,9,12,13-¹³C]-melatonin: 5-¹³CD₃O—C₆H₃C(¹³CD₂CD₂NHCO¹³CD₃)CH—NH
[13,13,13,9,9,10,10,12,12,12-D₁₀,10,12,13-¹³C]-melatonin: 5-CD₃O—C₆H₃C(CD₂¹³CD₂NHCO¹³CD₃)CH—NH
[9,9,10,10-D₄,9-¹³C]-melatonin: 5-CH₃O—C₆H₃C(¹³CD₂CD₂NHCOCH₃)CH—NH
[9,9,10,10-D₄,10-¹³C]-melatonin: 5-CH₃O—C₆H₃C(CD₂¹³CD₂NHCOCH₃)CH—NH
[1,1,1,2,2-D₅,1-¹³C]-rivastigmine tartrate: (S)—N-Ethyl-D₅,¹³C—N-methyl-3-[1-(dimethylamino)ethyl]-phenyl carbamate
[16,16,16-D₃,16-¹³C]-rivastigmine tartrate: (S)—N-Ethyl-N-methyl-3-[1-(dimethylamino-D₃,¹³C)ethyl]-phenyl carbamate
[13,13,13,12-D₄,13-¹³C]-rivastigmine tartrate: (S)—N-Ethyl-N-methyl-3-[1-(dimethylamino)ethyl-D₄,¹³C]-phenyl carbamate
[13,13,13,12-D₄,12-¹³C]-rivastigmine tartrate: (S)—N-Ethyl-N-methyl-3-[1-(dimethylamino)ethyl-D₄,¹³C]-phenyl carbamate
[16,16,16,15,15,15-D₆,15,16-¹³C₂]-rivastigmine tartrate: (S)—N-Ethyl-N-methyl-3-[1-(dimethylamino-D₆,¹³C₂)ethyl]-phenyl carbamate
[3,3-D₂,3-¹³C]-rasagiline: (R)—N-(D₂-prop-2-ynyl)-2,3-dihydro-1H-inden-1-amine
[14,14,14-D₃,14-¹³C]-methylphenidate: methyl-[D₃,¹³C]phenyl(piperidin-2-yl)acetate
[D₁₈,2-¹³C]-methylphenidate: D₁₈-methyl-[¹³C]phenyl(piperidin-2-yl)acetate-¹³C
[D₁₈,14-¹³C]-methylphenidate: D₁₈-methyl-[¹³C]phenyl(piperidin-2-yl)acetate-¹³C
[9,9,9-D₃,9-¹³C]-amphetamine: 1-phenylpropan-2-amine,3,3,3-D₃,3-¹³C
[9,9,9,1,2,2-D₆,1-¹³C]-amphetamine: 1-phenylpropan-2-amine,1,1,2,3,3,3-D₆,2-¹³C
[9,9,9,1,2,2-D₆,2-¹³C]-amphetamine: 1-phenylpropan-2-amine,1,1,2,3,3,3-D₆,1-¹³C
[9,9,9,1,2,2,4,5,6,7,8-D₁₁,1-¹³C]-amphetamine: 1-phenylpropan-2-amine,D₁₁,2-¹³C
[9,9,9,1,2,2,4,5,6,7,8-D₁₁,2-¹³C]-amphetamine: 1-phenylpropan-2-amine,D₁₁,1-¹³C
[9-D,9-¹³C]—(S)-2-amino-3-(5-hydroxy-1H-indol-3-yl)propenoic acid: 5-OHC₆H₃C(¹³CDC(NH₂)COOH)CHNH
[9-D,10-¹³C]—(S)-2-amino-3-(5-hydroxy-1H-indol-3-yl)propenoic acid: 5-OHC₆H₃C(CD¹³C(NH₂)COOH)CHNH
[6,4,3,1,9-D₅,10-¹³C]—(S)-2-amino-3-(5-hydroxy-1H-indol-3-yl)propenoic acid: 5-OHC₆D₃C(CD¹³C(NH₂)COOH)CDNH
[6,4,3,1,9-D₅,8-¹³C]—(S)-2-amino-3-(5-hydroxy-1H-indol-3-yl)propenoic acid: 5-OHC₆D₃¹³C(CDC(NH₂)COOH)CDNH
[3,4,4,5,5-D₅,3-¹³C]-2-amino-2-ene-5-(diaminomethylidene amino)pentanoic acid: ⁺NH₂═C(NH₂)NHCD₂CD₂¹³CDC(NH₂)CO₂H
[3,4,4,5,5-D₅,4-¹³C]-2-amino-2-ene-5-(diaminomethylidene amino)pentanoic acid: ⁺NH₂═C(NH₂)NHCD₂¹³CD₂CDC(NH₂)CO₂H
[3,4,4,5,5-D₅,5-¹³C]-2-amino-2-ene-5-(diaminomethylidene amino)pentanoic acid: ⁺NH₂═C(NH₂)NH¹³CD₂CD₂CDC(NH₂)CO₂H
[2,3,3,4,4,5-D₆,2-¹³C]-2-amino-5-(diaminomethylidene imino)pentanoic acid: ⁺NH₂C(NH₂)NCDCD₂CD₂¹³CD(NH₂)CO₂H
[2,3,3,4,4,5-D₆,3-¹³C]-2-amino-5-(diaminomethylidene imino)pentanoic acid: ⁺NH₂C(NH₂)NCDCD₂¹³CD₂CD(NH₂)CO₂H
[2,3,3,4,4,5-D₆,4-¹³C]-2-amino-5-(diaminomethylidene imino)pentanoic acid: ⁺NH₂C(NH₂)NCD¹³CD₂CD₂CD(NH₂)CO₂H
[2,3,3,4,4,5-D₆,4-¹³C]-2-amino-5-(diaminomethylidene imino)pentanoic acid: ⁺NH₂C(NH₂)N¹³CDCD₂CD₂CD(NH₂)CO₂H
[2,3,3,4,4,5-D₆,5-¹³C]-2-amino-5-(diaminomethylidene imino)pentanoic acid: ⁺NH₂C(NH₂)N¹³CDCD₂CD₂CD(NH₂)CO₂H
including any metabolite or derivative thereof.

41. A neurochemical agent according to claim 32, being in a hyperpolarized state.

42. A composition comprising a neurochemical agent according to claim 32.

43. A method for diagnosing and evaluating a condition or disease in a subject, said method comprising:
hyperpolarizing a neurochemical agent comprising at least one isotopically labeled carbon atom directly bonded to at least one deuterium atom;
administering to said subject an effective amount of hyperpolarized neurochemical agent;
monitoring said hyperpolarized neurochemical agent or any metabolite thereof;
thereby diagnosing said neurochemical condition or disease.

44. A method according to claim 43, wherein said monitoring is performed by means of magnetic resonance spectroscopy.

45. A method according to claim 43, wherein said subject is administered with consecutive doses of said hyperpolarized neurochemical agent.

46. A method according to claim 43, wherein said diagnosis and evaluation is performed during or after said subject is administered with at least one therapeutic agent.

47. A method according to claim 43, wherein said condition or disease is selected from Alzheimer's disease, Parkinson's diseases, depression, brain injury, dementia, mild cognitive impairment, affective disorders, serotonin syndrome, hyperserotonemia, neuroleptic malignant syndrome, schizophrenia, addiction, atherosclerosis and cancer.

48. A kit comprising at least one component containing at least one neurochemical agent comprising at least one isotopically labeled carbon atom directly bonded to at least one deuterium atom, means for administering said at least one agent and instructions for use.

49. A kit according to claim 48, for use in diagnosing and evaluating a neurochemical condition or disease.
